(19) United States
(12) Patent Application Publication
(10) Pub. No.: US 2025/0312340 A1
(43) Pub. Date: Oct. 9, 2025

(54) METHODS OF TREATING NEUROPATHIC PAIN

(71) Applicant: INTRA-CELLULAR THERAPIES, INC., Bedminster, NJ (US)

(72) Inventors: Robert DAVIS, San Diego, CA (US); Gretchen SNYDER, New York, NY (US); Kimberly VANOVER, New York, NY (US)

(21) Appl. No.: 19/239,540

(22) Filed: Jun. 16, 2025

Related U.S. Application Data
(63) Continuation of application No. 17/601,390, filed on Oct. 4, 2021, now abandoned.
(60) Provisional application No. 62/829,417, filed on Apr. 4, 2019.

(51) Int. Cl. A61K 31/4985; A61P 25/02

(52) U.S. Cl. CPC A61K 31/4985; A61P 25/02

(57) ABSTRACT

The invention relates to particular substituted heterocycle fused gamma-carbolines, in free, solid, pharmaceutically acceptable salt and/or substantially pure form as described herein, pharmaceutical compositions thereof, for use in methods for the treatment of neuropathic pain.

### CROSS-REFERENCE TO RELATED APPLICATIONS

This application is an international application claiming priority to, and the benefit of, U.S. provisional application Ser. No. 62/829,417, filed on Apr. 4, 2019, the contents of which are hereby incorporated by reference in its entirety.

### FIELD OF THE INVENTION

The invention relates to the use of particular substituted heterocycle fused gamma-carbolines, in free or pharmaceutically acceptable salt and/or substantially pure form as described herein, pharmaceutical compositions thereof, for the treatment and/or prevention of neuropathic pain.

### BACKGROUND OF THE INVENTION

Substituted heterocycle fused gamma-carbolines are known to be agonists or antagonists of 5-HT₂ receptors, particularly 5-HT_{2A} receptors, in treating central nervous system disorders. These compounds have been disclosed in U.S. Pat. Nos. 6,548,493; 7,238,690; 6,552,017; 6,713,471; 7,183,282; U.S. RE39680, and U.S. RE39679, as novel compounds useful for the treatment of disorders associated with 5-HT_{2A} receptor modulation such as obesity, anxiety, depression, psychosis, schizophrenia, sleep disorders, sexual disorders migraine, conditions associated with cephalic pain, social phobias, gastrointestinal disorders such as dysfunction of the gastrointestinal tract motility, and obesity. U.S. Pat. Nos. 8,309,722, and 7,081,455, also disclose methods of making substituted heterocycle fused gamma-carbolines and uses of these gamma-carbolines as serotonin agonists and antagonists useful for the control and prevention of central nervous system disorders such as addictive behavior and sleep disorders.

In addition, U.S. Pat. No. 8,598,119 discloses use of particular substituted heterocycle fused gamma-carbolines for the treatment of a combination of psychosis and depressive disorders as well as sleep, depressive and/or mood disorders in patients with psychosis or Parkinson's disease. In addition to disorders associated with psychosis and/or depression, this patent application discloses and claims use of these compounds at a low dose to selectively antagonize 5-HT_{2A} receptors without affecting or minimally affecting dopamine D2 receptors, thereby useful for the treatment of sleep disorders without the side effects associated with high occupancy of the dopamine D₂ pathways or side effects of other pathways (e.g., GABA_{A} receptors) associated with conventional sedative-hypnotic agents (e.g., benzodiazepines) including but not limited to the development of drug dependency, muscle hypotonia, weakness, headache, blurred vision, vertigo, nausea, vomiting, epigastric distress, diarrhea, joint pains, and chest pains. U.S. Pat. No. 8,648,077 also discloses methods of preparing toluenesulfonic acid addition salt crystals of these substituted heterocycle fused gamma-carbolines.

In addition, without being bound by theory, recent evidence shows that some of the aforementioned substituted fused heterocycle gamma carbolines may operate, in part, through NMDA receptor antagonism via mTOR1 signaling, in a manner similar to that of ketamine. Ketamine is a selective NMDA receptor antagonist. Ketamine acts through a system that is unrelated to the common psychogenic monoamines (serotonin, norepinephrine and dopamine), and this is a major reason for its much more rapid effects. Ketamine directly antagonizes extrasynaptic glutamatergic NMDA receptors, which also indirectly results in activation of AMPA-type glutamate receptors. The downstream effects involve the brain-derived neurotrophic factor (BDNF) and mTORC1 kinase pathways. Similar to ketamine, recent evidence suggests that compounds related to those of the present disclosure enhance both NMDA and AMPA-induced currents in rat medial prefrontal cortex pyramidal neurons via activation of D1 receptors, and that this is associated with increased mTORC1 signaling. International application PCT/US2018/043100 (WO 2019/023062, the contents of which are incorporated by reference in its entirety) discloses such effects for certain substituted fused heterocycle gamma-carbolines, and useful therapeutic indications related thereto.

U.S. Pat. No. 10,245,260 discloses additional novel fused heterocycle gamma carbolines. These new compounds were found to display serotonin receptor inhibition, SERT inhibition, and dopamine receptor modulation. However, these compounds were also unexpectedly found to show significant activity at mu-opiate receptors. Analogs of these novel compounds have also been disclosed, for example, in publications WO 2018/126140, WO 2018/126143, and WO 2019/23063, the contents of which are incorporated by reference in their entireties. Among the indications disclosed in these publications are, generally, the treatment of pain, neuropathic pain, and chronic pain.

For example, the Compound of Formula A, shown below, is a potent serotonin 5-HT_{2A} receptor antagonist and mu-opiate receptor partial, biased agonist. This compound also interacts with dopamine receptors, in particular dopamine D1 receptors.

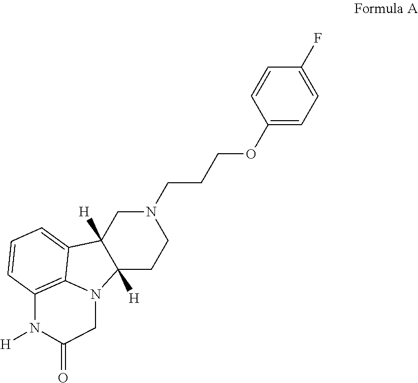 [SMILES: O=C1CN2c3c(cccc3[C@@H]3CN(CCCOc4ccc(F)cc4)CC[C@@H]32)N1]

It is also believed that the Compound of Formula A, via its D1 receptor activity, may also enhance NMDA and AMPA mediated signaling through the mTOR pathway. The Compound of Formula A is thus useful for the treatment or prophylaxis of central nervous system disorders, including opiate addiction, such as opiate use disorder.

Pain is the most common reason that patients seek medical care. See THE MERCK MANUAL OF DIAGNOSIS AND THERAPY 1965-85 (Merck Sharpe & Dohme 2018). Acute pain, which usually involves tissue injury, is caused by activation of peripheral pain receptors and their specific A delta and C sensory nerve fibers. Chronic pain caused by continuing tissue injury is believed to be caused by chronic stimulation of these same sensory pathways. However, in cases of neuropathic pain, there is no peripheral tissue injury, and the pain is caused by damage to or dysfunction of the nervous system itself (either the peripheral nerves or the central nervous system).

Neuropathic pain may be rooted in an underlying peripheral nerve injury or dysfunction. These include the mononeuropathies, such as carpal tunnel syndrome and radiculopathy, the plexopathies, such as nerve compression caused by tumors or herniated disks, and the polyneuropathies. The mechanisms behind neuropathic pain are still poorly understood, but may involve, in some cases, increased density of sodium channels on regenerating nerves.

Neuropathic pain may also be rooted in an underlying central neuropathic pain syndrome. These are thought to involve reorganization of central somatosensory processing pathways, including deafferentation pain and sympathetically maintained pain. Deafferentation pain is due to partial or complete interruption of peripheral or central afferent neural activity, such as in postherpetic neuralgia, pain after a central nervous system injury, and phantom limb pain (see after traumatic or non-traumatic [surgical] amputations). Sympathetically maintained pain depends on efferent sympathetic activity. Complex regional pain syndrome (CRPS) sometimes involves sympathetically maintained pain. Mechanisms may include abnormal sympathetic-somatic nerve connections (ephapses), local inflammatory changes and/or changes in the spinal cord.

Symptoms of neuropathic pain can vary, and may include dyesthesias (spontaneous or evoked burning pain, often with a superimposed lancinating component), hyperesthesia, allodynia (pain due to a previously non-noxious stimulus), and hyperpathia (particularly unpleasant, exaggerated pain response). Symptoms are long lasting, and when they are tied to a primary cause (such as acute injury), they outlast the resolution of the primary cause.

Current treatments for neuropathic pain have very limited success. While several classes of drug show some benefit, complete or near-complete relief is unlikely. Surprisingly, traditional analgesic medications, such as non-opioid analgesics (e.g., non-steroidal anti-inflammatory drugs, NSAIDs) and opioid analgesics, are not commonly prescribed because they lack significant efficacy and/or present too high of a risk of addiction (in the case of opioids). Instead, the most frequently prescribed medications for neuropathic pain are antidepressants and anticonvulsants. Commonly prescribed antidepressants include amitriptyline, desipramine, and duloxetine. Commonly prescribed anticonvulsants include carbamazepine, gabapentin, phenytoin, pregabalin and valproate. Each of these agents come with different side effects and potential abuse liabilities.

Thus, there is a need for agents with an improved ability to treat neuropathic pain that have reduced side effect liabilities.

### SUMMARY OF THE INVENTION

The present disclosure provides a method for the treatment of neuropathic pain, comprising administering to a patient in need thereof a Compound of Formula I, or a pharmaceutical composition thereof, wherein the Compound of Formula I is:

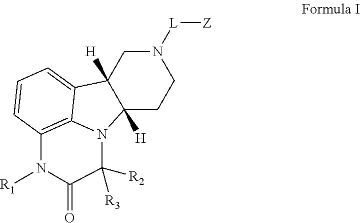

wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
R² and R³ are independently selected from H, D, C₁₋₆alkyl (e.g., methyl), C₁₋₆alkoxy (e.g., methoxy), halo (e.g., F), cyano, or hydroxy;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₆alkylamino or N-C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, or hydroxy;
Z is selected from aryl (e.g., phenyl) and heteroaryl (e.g., pyridyl, indazolyl, benzimidazolyl, benzisoxazolyl), wherein said aryl or heteroaryl is optionally substituted with one or more R⁴ moieties;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form).

In additional aspects, the present disclosure further provides use of a Compounds of the present disclosure, e.g., a Compound of Formula I, in the manufacture of a medicament for the treatment of neuropathic pain. The present disclosure further provides a Compound of the present disclosure, e.g., a Compound of Formula I, for use in the treatment of neuropathic pain.

### DETAILED DESCRIPTION OF THE INVENTION

In a first aspect, the present disclosure provides a method (Method 1) for the treatment of chronic pain and/or neuropathic pain, comprising administering to a patient in need thereof a Compound of Formula I, or a Pharmaceutical Composition I, I-A, I-B, I-C, or any of P.1-P.7 comprising a Compound of Formula I, wherein the Compound of Formula I is:

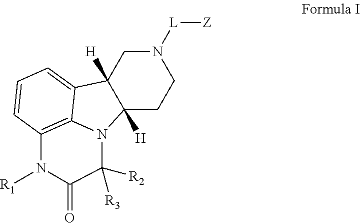

wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
R² and R³ are independently selected from H, D, C₁₋₆alkyl (e.g., methyl), C₁₋₆alkoxy (e.g., methoxy), halo (e.g., F), cyano, or hydroxy;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₆alkylamino or N-C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, or hydroxy;
Z is selected from aryl (e.g., phenyl) and heteroaryl (e.g., pyridyl, indazolyl, benzimidazolyl, benzisoxazolyl), wherein said aryl or heteroaryl is optionally substituted with one or more R⁴ moieties;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form);
wherein the pain is caused by a peripheral neuropathy (e.g., a mononeuropathy, a plexopathy, a radiculopathy, or a polyneuropathy) or is caused by a central neuropathy (e.g., deafferentation pain or sympathetically maintained pain, such as complex regional pain syndrome (CRPS)).

The present disclosure provides additional exemplary embodiments Method 1, including:
1.1 Method 1, comprising the compound of Formula I wherein R¹ is H;
1.2 Method 1, comprising the compound of Formula I wherein R¹ is C₁₋₆alkyl, e.g., methyl;
1.3 Method 1, comprising the compound of Formula I wherein R¹ is —C(O)—O—C(R^{a})(R^{b})(R^{c});
1.4 Method 1.3, comprising the compound of Formula I wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or Chialkyl, C₁₂alkyl, C₁₃alkyl, C₁₄alkyl, Cisalkyl or C₁₆alkyl;
1.5 Method 1.3, comprising the compound of Formula I wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or Chialkyl, C₁₂alkyl, C₁₃alkyl, C₁₄alkyl, Cisalkyl or C₁₆alkyl;
1.6 Method 1.3, comprising the compound of Formula I wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or Cuialkyl, C₁₂alkyl, C₁₃alkyl, C₁₄alkyl, Cisalkyl or C₁₆alkyl;
1.7 Method 1.3, comprising the compound of Formula I wherein R^{a}, R^{b} and R^{c} are each H;
1.8 Method 1.3, comprising the compound of Formula I wherein R^{a} and R^{b} are H and R^{c} is C₁₀₋₁₄alkyl (e.g., R^{c} is CH₃(CH₂)₁₀ or CH₃(CH₂)₁₄);
1.9 Method 1, comprising the compound of Formula I wherein R¹ is —C(O)—O—CH₂—O—C(R^{a})(R^{b})(R^{c});
1.10 Method 1.9, comprising the compound of Formula I wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or Cualkyl, C₁₂alkyl, C₁₃alkyl, C₁₄alkyl, Cisalkyl or C₁₆alkyl;
1.11 Method 1.9, comprising the compound of Formula I wherein R^{a} and R^{b} are H and R^{c} is C₁₋₂₄alkyl, e.g., C₁₋₂₀alkyl, C₅₋₂₀alkyl, C₉₋₁₈alkyl, C₁₀₋₁₆alkyl, or Chialkyl, C₁₂alkyl, C₁₃alkyl, C₁₄alkyl, C₁₅salkyl or C₁₆alkyl;
1.12 Method 1.9, comprising the compound of Formula I wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.13 Method 1.9, comprising the compound of Formula I wherein R^{a}, R^{b} and R^{c} are each H;
1.14 Method 1, comprising the compound of Formula I wherein R¹ is —C(R⁶)(R⁷)—O—C(O)—R⁸, and R⁸ is —C(R^{a})(R^{b})(R^{c});
1.15 Method 1, comprising the compound of Formula I wherein R¹ is —C(R⁶)(R⁷)—O—C(O)—R⁸, and R⁸ is —O—C(R^{a})(R^{b})(R^{c});
1.16 Method 1.14 or 1.15, comprising the compound of Formula I wherein R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.17 Method 1.14 or 1.15, comprising the compound of Formula I wherein R^{a} and R^{b} are Hand R^{c} is C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.18 Method 1.14 or 1.15, comprising the compound of Formula I wherein R^{a}, R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.19 Method 1.14 or 1.15, comprising the compound of Formula I wherein R^{a}, R^{b} and R^{c} are each H;
1.20 Any of Methods 1.14-1.19, comprising the compound of Formula I wherein R⁶ is H, and R⁷ is C₁₋₃alkyl (e.g., R⁷is methyl or isopropyl), and R⁸ is C₁₀₋₁₄alkyl (e.g., R⁸ is CH₃(CH₂)₁₀ or CH₃(CH₂)₁₄);
1.21 Method 1, comprising the compound of Formula I wherein R¹ is —C (R⁶)(R⁷)—O—C(O)—R⁸, and R⁸ is —N(R^{d})(R^{e});
1.22 Method 1.21, comprising the compound of Formula I wherein R^{d} is H and R^{e} is independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.23 Method 1.21, comprising the compound of Formula I wherein R^{d} and R^{e} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.24 Method 1.21, comprising the compound of Formula I wherein R^{d} and R^{e} are each H;
1.25 Any of Methods 1.14-1.24, comprising the compound of Formula I wherein R⁶ is H and R⁷ is H;
1.26 Any of Methods 1.14-1.24, comprising the compound of Formula I wherein R⁶ is C₁₋₆alkyl and R⁷ is C₁₋₆alkyl;
1.27 Any of Methods 1.14-1.24, comprising the compound of Formula I wherein R⁶ is H and R⁷ is C₁₋₆alkyl;
1.28 Any of Methods 1.14-1.24, comprising the compound of Formula I wherein R⁶ is H and R⁷ is carboxy;
1.29 Any of Methods 1.14-1.24, comprising the compound of Formula I wherein R⁶ is H and R⁷ is C₁₋₆alkoxycarbonyl, e.g., ethoxycarbonyl or methoxycarbonyl;
1.30 Method 1, or any of 1.1-1.29, comprising the compound of Formula I wherein R² and R³ are H;
1.31 Method 1, or any of 1.1-1.29, comprising the compound of Formula I wherein R² is H and R³ is D;
1.32 Method 1, or any of 1.1-1.29, comprising the compound of Formula I wherein R² and R³ are D;
1.33 Method 1, or any of 1.1-1.32, comprising the compound of Formula I wherein L is C₁₋₆alkylene (e.g., ethylene, propylene, or butylene), C₁₋₆alkoxy (e.g., propoxy), C₂₋₃alkoxyC₁₋₃alkylene (e.g., CH₂CH₂OCH₂) C₁₋₆alkylamino (e.g., propylamino or N-methylpropylamino), or C₁₋₆alkylthio (e.g., —CH₂CH₂CH₂S—), optionally substituted with one or more R⁴ moieties;
1.34 Method 1.33, comprising the compound of Formula I wherein L is unsubstituted C₁₋₆alkylene (e.g., ethylene, propylene, or butylene);
1.35 Method 1.33, comprising the compound of Formula I wherein L is C₁₋₆alkylene (e.g., ethylene, propylene, or butylene), substituted with one or more R⁴ moieties;
1.36 Method 1.33, comprising the compound of Formula I wherein L is unsubstituted C₁₋₆alkyoxy (e.g., propoxy or butoxy);
1.37 Method 1.33, comprising the compound of Formula I wherein L is C₁₋₆alkoxy (e.g., propoxy or butoxy), substituted with one or more R⁴ moieties;
1.38 Method 1.33, comprising the compound of Formula I wherein L is unsubstituted C₂₋₃alkoxyC₁₋₃alkylene (e.g., CH₂CH₂OCH₂);
1.39 Method 1.33, comprising the compound of Formula I wherein L is C₂₋₃alkoxyC₁₋₃salkylene (e.g., CH₂CH₂OCH₂), substituted with one or more R⁴ moieties;
1.40 Method 1, or any of 1.1-1.39, comprising the compound of Formula I wherein R¹, R² and R³ are each H;
1.41 Method 1, or any of 1.1-1.40, comprising the compound of Formula I wherein L is —(CH₂)ₙ—X—, and wherein n is an integer selected from 2, 3 and 4, and X is selected from —O—, —S—, —NH—, —N(C₁₋₆alkyl)—, and CH₂;
1.42 Method 1.41, comprising the compound of Formula I wherein L is —(CH₂)ₙ—X—, and wherein n is an integer selected from 2, 3 and 4, and X is —O—;
1.43 Method 1.41, comprising the compound of Formula I wherein L is —(CH₂)ₙ—X—, and wherein n is 3, and X is selected from —O—, —S—, —NH— and —N(C₁₋₆alkyl)-(e.g., —N(CH₃)—);
1.44 Method 1.41, comprising the compound of Formula I wherein L is —(CH₂)ₙ—X—, and wherein n is 3, and X is CH₂;
1.45 Method 1, or any of 1.1-1.44, comprising the compound of Formula I wherein Z is aryl (e.g., phenyl), optionally substituted with one or more R⁴ moieties;
1.46 Method 1.45, comprising the compound of Formula I wherein Z is aryl (e.g., phenyl), substituted with one or more R⁴ moieties;
1.47 Method 1.46, comprising the compound of Formula I wherein Z is phenyl substituted with one, two, three or four R⁴ moieties;
1.48 Method 1.46, comprising the compound of Formula I wherein the one, two three or four R⁴ moieties are independently selected from halo (e.g., fluoro, chloro, bromo or iodo) and cyano;
1.49 Method 1.46, comprising the compound of Formula I wherein Z is phenyl substituted with one R⁴ moiety selected from halo (e.g., fluoro, chloro, bromo or iodo) and cyano (e.g., Z is 4-fluorophenyl, or 4-chlorophenyl, or 4-cyanophenyl);
1.50 Method 1.46, comprising the compound of Formula I wherein Z is phenyl substituted with one fluoro (e.g., 2-fluorophenyl, 3-fluorophenyl or 4-flourophenyl);
1.51 Method 1.46, comprising the compound of Formula I wherein Z is 4-fluoroophenyl;
1.52 Method I, or any of 1.1-1.44, comprising the compound of Formula I wherein Z is heteroaryl (e.g., pyridyl, indazolyl, benzimidazolyl, benzisoxazolyl), optionally substituted with one or more R⁴ moieties;

1.53 Method 1.52, comprising the compound of Formula I wherein said heteroaryl is a monocyclic 5-membered or 6-membered heteroaryl (e.g., pyridyl, pyrimidyl, pyrazinyl, thiophenyl, pyrrolyl, thiophenyl, furanyl, imidazolyl, oxazolyl, isoxazolyl, thiazolyl);
1.54 Method 1.53, comprising the compound of Formula I wherein said heteroaryl is selected from pyridyl, pyrimidinyl and pyrazinyl;
1.55 Method 1.52, comprising the compound of Formula I wherein said heteroaryl is a bicyclic 9-membered or 10-membered heteroaryl (e.g., indolyl, isoindolyl, benzfuranyl, benzthiophenyl, indazolyl, benzimidazolyl, benzoxazolyl, benzisoxazolyl, benzthiazolyl, quinolinyl, isoquinolinyl, quinoxalinyl, quinazolinyl, benzodioxolyl, 2-oxo-tetrahydroquinolinyl);
1.56 Method 1.55, comprising the compound of Formula I wherein said heteroaryl is selected from indazolyl, benzisoxazolyl, quinolinyl, benzodioxolyl, and 2-oxo-tetrahydroquinolinyl);
1.57 Method 1.55, comprising the compound of Formula I wherein said heteroaryl is selected from indazolyl, benzisoxazolyl, and quinolinyl);
1.58 Any of Methods 1.52-1.57, comprising the compound of Formula I wherein said heteroaryl is substituted with one, two, three or four R⁴ moieties;
1.59 Method 1.58, comprising the compound of Formula I wherein the one, two three or four R⁴ moieties are independently selected from halo (e.g., fluoro, chloro, bromo or iodo), cyano, hydroxy, or C₁₋₆alkoxy (e.g., methoxy);
1.60 Method 1.58 or 1.59, comprising the compound of Formula I wherein said heteroaryl is substituted with one R⁴ moiety selected from halo (e.g., fluoro, chloro, bromo or iodo) and cyano (e.g., said heteroaryl is 6-fluoro-3-indazolyl, 6-chloro-3-indazolyl, 6-fluoro-3-benzisoxazolyl, or 5-chloro-3-benzisoxazolyl);
1.61 Method 1, or any of 1.1-1.60, comprising the compound of Formula I wherein the compound is selected from the group consisting of:

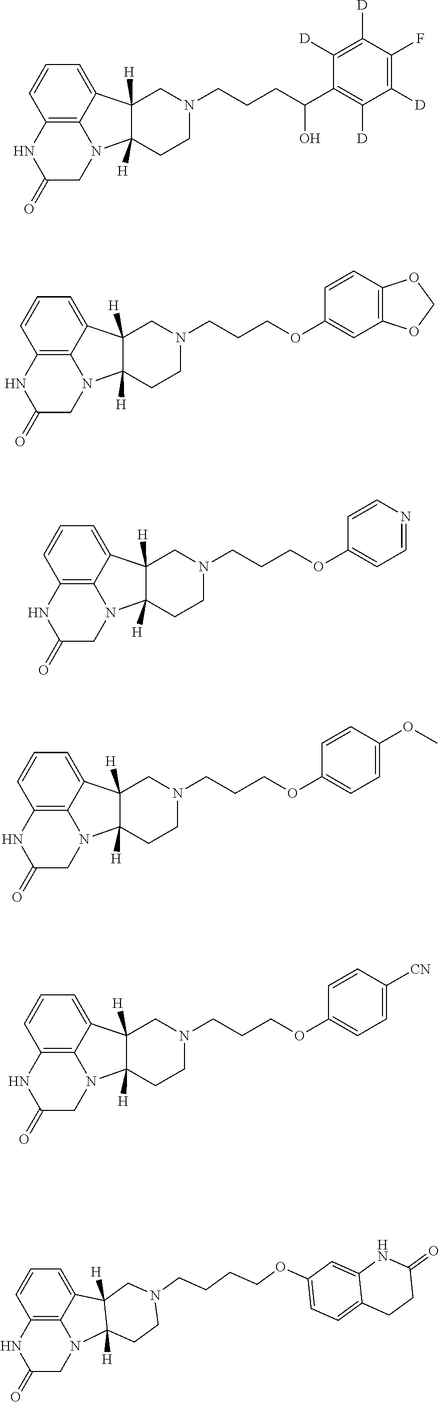 [SMILES: [2H]c1c([2H])c(C(O)CCCN2CC[C@H]3[C@@H](C2)c2cccc4c2N3CC(=O)N4)c([2H])c([2H])c1F ; O=C1CN2c3c(cccc3[C@@H]3CN(CCCOc4ccc5c(c4)OCO5)CC[C@@H]32)N1 ; O=C1CN2c3c(cccc3[C@@H]3CN(CCCOc4ccncc4)CC[C@@H]32)N1 ; COc1ccc(OCCCN2CC[C@H]3[C@@H](C2)c2cccc4c2N3CC(=O)N4)cc1 ; N#Cc1ccc(OCCCN2CC[C@H]3[C@@H](C2)c2cccc4c2N3CC(=O)N4)cc1 ; O=C1CCc2ccc(OCCCCN3CC[C@H]4[C@@H](C3)c3cccc5c3N4CC(=O)N5)cc2N1]

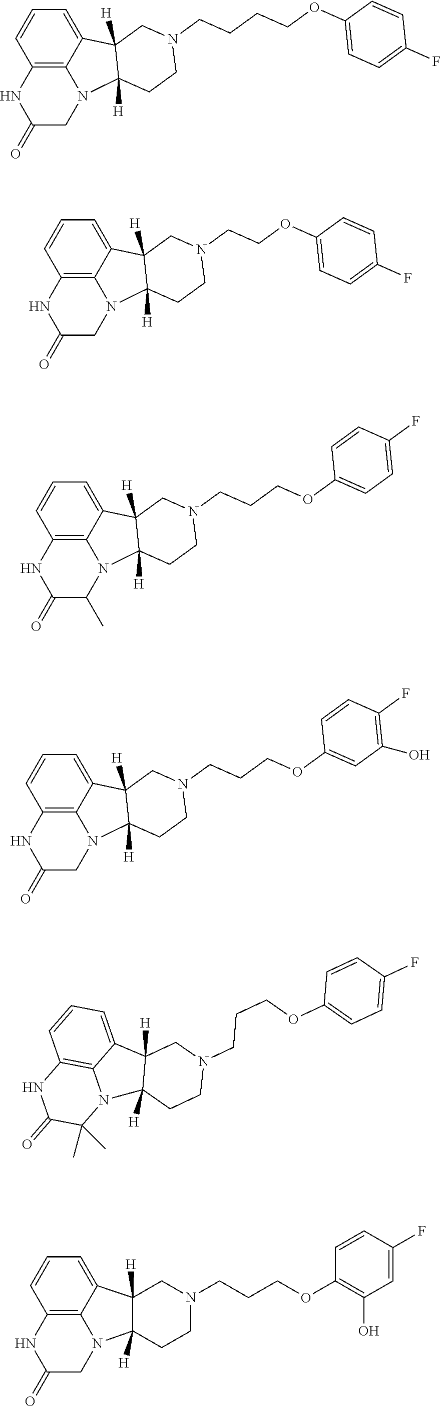 [SMILES: O=C1CN2c3c(cccc3[C@@H]3CN(CCCCOc4ccc(F)cc4)CC[C@@H]32)N1 ; O=C1CN2c3c(cccc3[C@@H]3CN(CCOc4ccc(F)cc4)CC[C@@H]32)N1 ; CC1C(=O)Nc2cccc3c2N1[C@H]1CCN(CCCOc2ccc(F)cc2)C[C@@H]31 ; O=C1CN2c3c(cccc3[C@@H]3CN(CCCOc4ccc(F)c(O)c4)CC[C@@H]32)N1 ; CC1(C)C(=O)Nc2cccc3c2N1[C@H]1CCN(CCCOc2ccc(F)cc2)C[C@@H]31 ; O=C1CN2c3c(cccc3[C@@H]3CN(CCCOc4ccc(F)cc4O)CC[C@@H]32)N1]

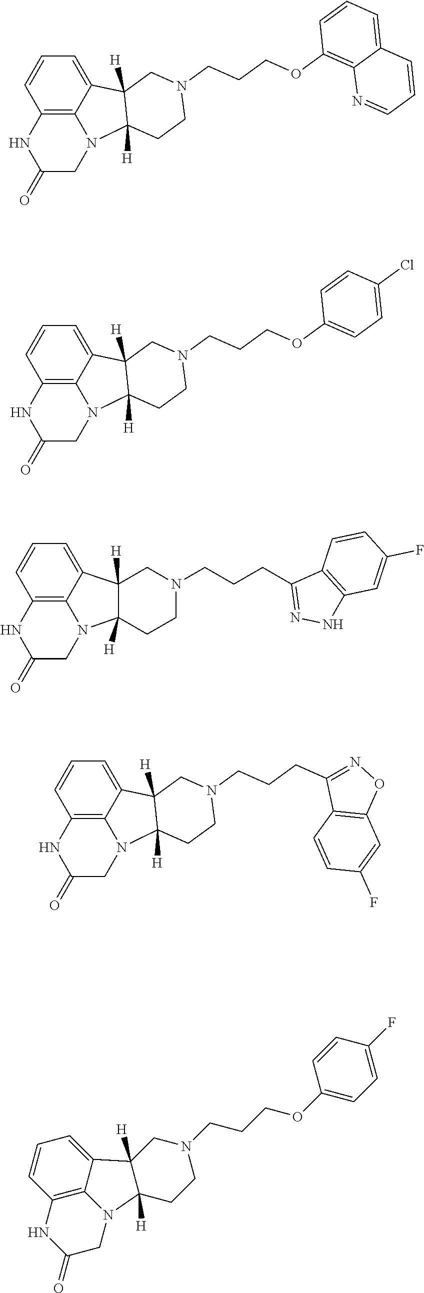 [SMILES: O=C1CN2c3c(cccc3[C@@H]3CN(CCCOc4cccc5cccnc45)CC[C@@H]32)N1 ; O=C1CN2c3c(cccc3[C@@H]3CN(CCCOc4ccc(Cl)cc4)CC[C@@H]32)N1 ; O=C1CN2c3c(cccc3[C@@H]3CN(CCCc4n[nH]c5cc(F)ccc45)CC[C@@H]32)N1 ; O=C1CN2c3c(cccc3[C@@H]3CN(CCCc4noc5cc(F)ccc45)CC[C@@H]32)N1 ; O=C1CN2c3c(cccc3[C@@H]3CN(CCCOc4ccc(F)cc4)CC[C@@H]32)N1]

each independently in free, or pharmaceutically acceptable salt form;
1.62 Method 1, or any of 1.1-1.60, comprising the compound of Formula I wherein the compound is selected from the group consisting of:

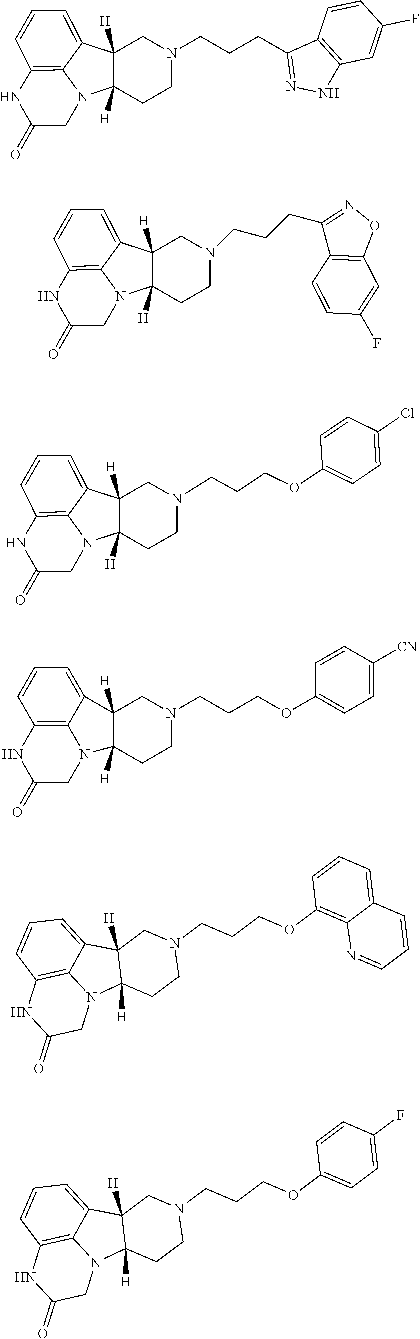 [SMILES: O=C1CN2c3c(cccc3[C@@H]3CN(CCCc4n[nH]c5cc(F)ccc45)CC[C@@H]32)N1 ; O=C1CN2c3c(cccc3[C@@H]3CN(CCCc4noc5cc(F)ccc45)CC[C@@H]32)N1 ; O=C1CN2c3c(cccc3[C@@H]3CN(CCCOc4ccc(Cl)cc4)CC[C@@H]32)N1 ; N#Cc1ccc(OCCCN2CC[C@H]3[C@@H](C2)c2cccc4c2N3CC(=O)N4)cc1 ; O=C1CN2c3c(cccc3[C@@H]3CN(CCCOc4cccc5cccnc45)CC[C@@H]32)N1 ; O=C1CN2c3c(cccc3[C@@H]3CN(CCCOc4ccc(F)cc4)CC[C@@H]32)N1]

each independently in free or pharmaceutically acceptable salt form;
1.63 Method 1, or any of 1.1-1.60, comprising the compound of Formula I wherein the compound is selected from the group consisting of:

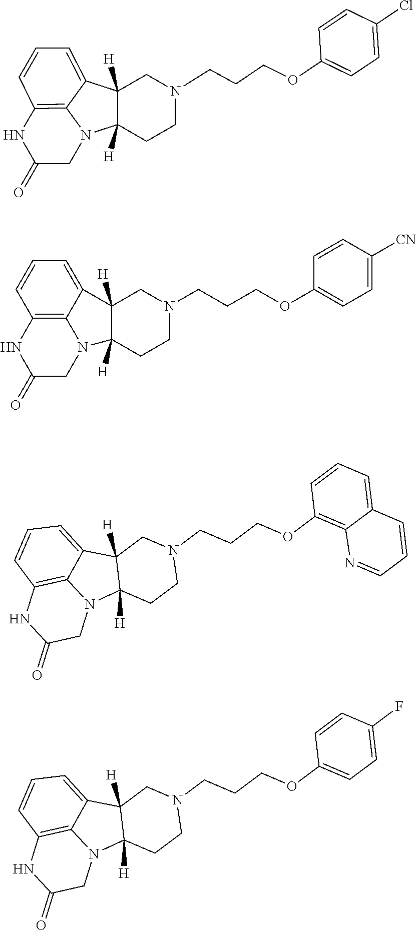 [SMILES: O=C1CN2c3c(cccc3[C@@H]3CN(CCCOc4ccc(Cl)cc4)CC[C@@H]32)N1 ; N#Cc1ccc(OCCCN2CC[C@H]3[C@@H](C2)c2cccc4c2N3CC(=O)N4)cc1 ; O=C1CN2c3c(cccc3[C@@H]3CN(CCCOc4cccc5cccnc45)CC[C@@H]32)N1 ; O=C1CN2c3c(cccc3[C@@H]3CN(CCCOc4ccc(F)cc4)CC[C@@H]32)N1]

each independently in free or pharmaceutically acceptable salt form;
1.64 Method 1, or any of 1.1-1.61, comprising the compound of Formula I wherein the compound is

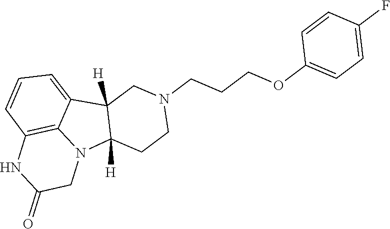 [SMILES: O=C1CN2c3c(cccc3[C@@H]3CN(CCCOc4ccc(F)cc4)CC[C@@H]32)N1]

in free or pharmaceutically acceptable salt form;
1.65 Method 1, or any of 1.1-1.64, comprising the compound of Formula I in free form;
1.66 Method 1, or any of 1.1-1.64, comprising the compound of Formula I in salt form, e.g., pharmaceutically acceptable salt form;
1.67 Method 1, or any of 1.1-1.64, comprising the compound of Formula I wherein the compound is in acid addition salt form, for example, wherein the acid is hydrochloric, toluenesulfonic, glutamic, tartaric, malic or ascorbic acid;
1.68 Method 1, or any of 1.1-1.67, comprising the compound of Formula I in substantially pure diastereomeric form (i.e., substantially free from other diastereomers);
1.69 Method 1, or any of 1.1-1.67, comprising the compound of Formula I having a diastereomeric excess of greater than 70%, preferably greater than 80%, more preferably greater than 90% and most preferably greater than 95%;
1.70 Method 1, or any of 1.1-1.69, comprising the compound of Formula I in solid form, e.g., in crystal form;
1.71 Method 1, or any of 1.1-1.70, comprising the compound of Formula I in isolated or purified form (e.g., in at least 90% pure form, or at least 95% or at least 98% or at least 99%);
1.72 Method 1 or any of 1.1-1.71, wherein the compound of Formula I is administered in the form of a pharmaceutical composition comprising the compound of Formula I in admixture with a pharmaceutically acceptable diluent or carrier;
1.73 Method 1.72, wherein the compound of Formula I is in pharmaceutically acceptable salt form in admixture with a pharmaceutically acceptable diluent or carrier;
1.74 Method 1.72 or 1.73, wherein the pharmaceutical composition is a sustained release or delayed release formulation, e.g., according to Pharmaceutical Composition 1-A as described herein;
1.75 Method 1.72, 1.73 or 1.74, wherein the pharmaceutical composition comprises the Compound of Formula I in a polymeric matrix, e.g., according to Pharmaceutical Composition 1-B as described herein;
1.76 Any of Methods 1.72-1.75, wherein the pharmaceutical composition is formulated as an osmotic controlled release oral delivery system, e.g., according to Pharmaceutical Composition 1-C or any of P.1 to P.7, as described herein;
1.77 Any of Methods 1.72-1.76, wherein the pharmaceutical composition is in the form of a tablet or capsule;
1.78 Any of Methods 1.72-1.77, wherein the pharmaceutical composition is formulated for oral, sublingual, or buccal administration;
1.79 Any of Methods 1.72-1.78, wherein the pharmaceutical composition is a rapidly-dissolving oral tablet (e.g., a rapidly dissolving sublingual tablet);
1.80 Any of Methods 1.72-1.76, wherein the pharmaceutical composition is formulated for intranasal or intrapulmonary administration (e.g., as an aerosol, mist, or powder for inhalation);
1.81 Any of Methods 1.72-1.75, wherein the pharmaceutical composition is formulated for administration by injection, for example, as a sterile aqueous solution;
1.82 Method 1.81, wherein the pharmaceutical composition is formulated for intravenous, intrathecal, intramuscular, subcutaneous or intraperitoneal injection.

As used herein, the term “Compound of the present disclosure” refers any of the compounds described in Method 1 or the compounds described in any of the embodiments of Methods 1.1 to 1.71.

In some embodiments, Method 1 comprises the administration of a Compound of the present disclosure in the form of a for a sustained or delayed release formulation (Pharmaceutical Composition 1-A), e.g., a depot formulation. In some embodiments, the Compound of Formula I or as described in any of Methods 1.1-1.71 is provided, preferably in free or pharmaceutically acceptable salt form, in admixture with a pharmaceutically acceptable diluent or carrier, in the form of an injectable depot, which provides sustained or delayed release of the compound.

In a particular embodiment, the Pharmaceutical Composition 1-A comprises a Compound of Formula I, or any Compound of the present disclosure, in free base or pharmaceutically acceptable salt form, optionally in crystal form, wherein the compound has been milled to, or the compound crystallized to, a microparticle or nanoparticle size, e.g., particles or crystals having a volume-based particle size (e.g., diameter or Dv50) of 0.5 to 100 microns, for example, for example, 5-30 microns, 10-20 microns, 20-100 microns, 20-50 microns or 30-50 microns. Such particles or crystals may be combined with a suitable pharmaceutically acceptable diluent or carrier, for example water, to form a depot formulation for injection. For example, the depot formulation may be formulated for intramuscular or subcutaneous injection with a dosage of drug suitable for 4 to 6 weeks of treatment. In some embodiments, the particles or crystals have a surface area of 0.1 to 5 m²/g, for example, 0.5 to 3.3 m²/g or from 0.8 to 1.2 m²/g.

In another embodiment, the present disclosure provides a Pharmaceutical Composition I-B, which is Pharmaceutical Composition I, wherein the Compound of Formula I (or any Compound of the present disclosure) is in a polymeric matrix. In one embodiment, the Compound of the present disclosure is dispersed or dissolved within the polymeric matrix. In a further embodiment, the polymeric matrix comprises standard polymers used in depot formulations such as polymers selected from a polyester of a hydroxyfatty acid and derivatives thereof, or a polymer of an alkyl alpha-cyanoacrylate, a polyalkylene oxalate, a polyortho ester, a polycarbonate, a polyortho-carbonate, a polyamino acid, a hyaluronic acid ester, and mixtures thereof. In a further embodiment, the polymer is selected from a group consisting of polylactide, poly d,1-lactide, poly glycolide, or PLGA, including any PLGA of 50:50 to 90:10 ratio of lactic to glycolic units (e.g., 50:50 to 75:25), such as PLGA 50:50, PLGA 85:15 and PLGA 90:10 polymer. In another embodiment, the polymer is selected form poly (glycolic acid), poly-D,L-lactic acid, poly-L-lactic acid, copolymers of the foregoing, poly(aliphatic carboxylic acids), copolyoxalates, polycaprolactone, polydioxanone, poly(ortho carbonates), poly(acetals), poly(lactic acid-caprolactone), polyortho esters, poly(glycolic acid-caprolactone), polyanhydrides, and natural polymers including albumin, casein, and waxes, such as, glycerol mono-and distearate, and the like. In a preferred embodiment, the polymeric matrix comprises poly (d,1-lactide-co-glycolide).

The Pharmaceutical Composition I-B is particularly useful for sustained or delayed release, wherein the Compound of the present disclosure is released upon degradation of the polymeric matrix. These Compositions may be formulated for controlled-and/or sustained-release of the Compounds of the present disclosure (e.g., as a depot composition) over a period of up to 180 days, e.g., from about 14 to about 30 to about 180 days. For example, the polymeric matrix may degrade and release the Compounds of the present disclosure over a period of about 30, about 60 or about 90 days. In another example, the polymeric matrix may degrade and release the Compounds of the present disclosure over a period of about 120, or about 180 days.

In still another embodiment, the Pharmaceutical Composition I or I-A or I-B may be formulated for administration by injection, for example, as a sterile aqueous solution.

In another embodiment, the present disclosure provides a Pharmaceutical Composition (Pharmaceutical Composition I-C) comprising a Compound of Formula I (or any Compound of the present disclosure) as hereinbefore described, in an osmotic controlled release oral delivery system (OROS), which is described in US 2001/0036472 and US 2009/0202631, the contents of each of which applications are incorporated by reference in their entirety. Therefore in one embodiment, the present disclosure provides a pharmaceutical composition or device comprising (a) a gelatin capsule containing a Compound of any of Formulae I in free or pharmaceutically acceptable salt form, optionally in admixture with a pharmaceutically acceptable diluent or carrier; (b) a multilayer wall superposed on the gelatin capsule comprising, in outward order from the capsule: (i) a barrier layer, (ii) an expandable layer, and (iii) a semipermeable layer; and (c) and orifice formed or formable through the wall (Pharmaceutical Composition P.1).

In another embodiment, the invention provides a pharmaceutical composition comprising a gelatin capsule containing a liquid, the Compound of Formula I (or any Compound of the present disclosure) in free or pharmaceutically acceptable salt form, optionally in admixture with a pharmaceutically acceptable diluent or carrier, the gelatin capsule being surrounded by a composite wall comprising a barrier layer contacting the external surface of the gelatin capsule, an expandable layer contacting the barrier layer, a semi-permeable layer encompassing the expandable layer, and an exit orifice formed or formable in the wall (Pharmaceutical Composition P.2).

In still another embodiment, the invention provides a composition comprising a gelatin capsule containing a liquid, the Compound of Formula I (or any Compound of the present disclosure) in free or pharmaceutically acceptable salt form, optionally in admixture with a pharmaceutically acceptable diluent or carrier, the gelatin capsule being surrounded by a composite wall comprising a barrier layer contacting the external surface of the gelatin capsule, an expandable layer contacting the barrier layer, a semipermeable layer encompassing the expandable layer, and an exit orifice formed or formable in the wall, wherein the barrier layer forms a seal between the expandable layer and the environment at the exit orifice (Pharmaceutical Composition P.3).

In still another embodiment, the invention provides a composition comprising a gelatin capsule containing a liquid, the Compound of Formula I (or any Compound of the present disclosure) in free or pharmaceutically acceptable salt form, optionally in admixture with a pharmaceutically acceptable diluent or carrier, the gelatin capsule being surrounded by a barrier layer contacting the external surface of the gelatin capsule, an expandable layer contacting a portion of the barrier layer, a semi-permeable layer encompassing at least the expandable layer, and an exit orifice formed or formable in the dosage form extending from the external surface of the gelatin capsule to the environment of use (Pharmaceutical Composition P.4). The expandable layer may be formed in one or more discrete sections, such as for example, two sections located on opposing sides or ends of the gelatin capsule.

In a particular embodiment, the Compound of the present disclosure in the Osmotic-controlled Release Oral Delivery System (i.e., in Composition P.1-P.4) is in a liquid formulation, which formulation may be neat, liquid active agent, liquid active agent in a solution, suspension, emulsion or self-emulsifying composition or the like.

Further information on Osmotic-controlled Release Oral Delivery System composition including characteristics of the gelatin capsule, barrier layer, an expandable layer, a semi-permeable layer; and orifice may be found in US 2001/0036472, the contents of which are incorporated by reference in their entirety.

Other Osmotic-controlled Release Oral Delivery System for the Compound of Formula I (or any Compound of the present disclosure) or the Pharmaceutical Composition of the present disclosure may be found in US 2009/0202631, the contents of which are incorporated by reference in their entirety. Therefore, in another embodiment, the invention provides a composition or device comprising (a) two or more layers, said two or more layers comprising a first layer and a second layer, said first layer comprises the Compound of Formulas I et seq., in free or pharmaceutically acceptable salt form, optionally in admixture with a pharmaceutically acceptable diluent or carrier, said second layer comprises a polymer; (b) an outer wall surrounding said two or more layers; and (c) an orifice in said outer wall (Pharmaceutical Composition P.5).

Pharmaceutical Composition P.5 preferably utilizes a semi-permeable membrane surrounding a three-layer-core: in these embodiments, the first layer is referred to as a first drug layer and contains low amounts of drug (e.g., the Compound of Formulas I et seq.) and an osmotic agent such as salt, the middle layer referred to as the second drug layer contains higher amounts of drug, excipients and no salt; and the third layer referred to as the push layer contains osmotic agents and no drug (Pharmaceutical Composition P.6). At least one orifice is drilled through the membrane on the first drug layer end of the capsule-shaped tablet.

Pharmaceutical Composition P.5 or P.6 may comprise a membrane defining a compartment, the membrane surrounding an inner protective subcoat, at least one exit orifice formed or formable therein and at least a portion of the membrane being semi-permeable; an expandable layer located within the compartment remote from the exit orifice and in fluid communication with the semi-permeable portion of the membrane; a first drug layer located adjacent the exit orifice; and a second drug layer located within the compartment between the first drug layer and the expandable layer, the drug layers comprising the Compound of the present disclosure in free or pharmaceutically acceptable salt thereof (Pharmaceutical Composition P.7). Depending upon the relative viscosity of the first drug layer and second drug layer, different release profiles are obtained. It is imperative to identify the optimum viscosity for each layer. In the present invention, viscosity is modulated by addition of salt, sodium chloride. The delivery profile from the core is dependent on the weight, formulation and thickness of each of the drug layers.

In a particular embodiment, the invention provides Pharmaceutical Composition P.7 wherein the first drug layer comprises salt and the second drug layer does not contain salt. Pharmaceutical Composition P.5-P.7 may optionally comprise a flow-promoting layer between the membrane and the drug layers.

Pharmaceutical Compositions P.1-P.7 will generally be referred to as Osmotic-controlled Release Oral Delivery System Composition.

In further embodiments of the first aspect, the present disclosure provides further embodiments of Method 1 as follows:
1.83 Method 1 or any of Methods 1.1-1.82, wherein the pain is a chronic pain.
1.84 Method 1 or any of Methods 1.1-1.82, wherein the pain is a neuropathic pain.
1.85 Method 1.83 or 1.84, wherein the pain is a chronic neuropathic pain.
1.86 Method 1 or any of Methods 1.1-1.85, wherein the pain is caused by a mononeuropathy (e.g., single mononeuropathy), such as a focal mononeuropathy, a pressure mononeuropathy, or an entrapment mononeuropathy (e.g., carpal tunnel syndrome);
1.87 Method 1 or any of Methods 1.1-1.85, wherein the pain is caused by a radiculopathy, e.g., caused by a herniated spinal disk, or caused by diabetic ischemia; 11.88 Method 1 or any of Methods 1.1-1.85, wherein the pain is caused by a plexopathy, such as, a plexopathy caused by nerve compression, e.g., nerve compression by a neuroma, tumor, or herniated disk;
1.89 Method 1 or any of Methods 1.1-1.85, wherein the pain is caused by a multiple mononeuropathy or a polyneuropathy, e.g., diabetic polyneuropathy;
1.90 Method 1 or any of Methods 1.1-1.85, wherein the pain is caused by a central neuropathic pain syndrome, such as deafferentation pain or complex regional pain syndrome (CRPS), or by fibromyalgia;
1.91 Method 1 or any of Methods 1.1-1.85, wherein the pain is caused by postherpetic neuralgia (PHN) or by fibromyalgia;
1.92 Method 1 or any of Methods 1.1-1.85, wherein the pain is caused by drug- induced neurotoxicity (e.g., by doxorubicin, etoposide, gemcitabine, ifosfamide, interferon alfa, platinum chemotherapeutics (e.g., cisplatin, carboplatin, oxaliplatin, nedaplatin, triplatin, phenanthriplatin, picoplatin, satraplatin), or vinca alkaloids (e.g., vinblastine, vincristine, vindesine, vinorelbine, or vinpocetin), or anti-retroviral nucleosides (e.g., didanosine, stavudine, zalcitabine));
1.93 Any of methods 1.83-1.92, wherein the neuropathy is an axonal neuropathy (i.e., an axonopathy);
1.94 Method 1 or any of Methods 1.1-1.93 wherein the patient has fibromyalgia, diabetes, human immunodeficiency virus (HIV) infection or acquired immune deficiency syndrome (AIDS), or cancer;
1.95 Method 1 or any of Methods 1.1-1.93 wherein the patient is undergoing concurrent treatment or has had past treatment with an anti-retroviral nucleoside, a platinum-based anti-neoplastic, or a vinca alkaloid anti-neoplastic);
1.96 Method 1 or any of Methods 1.1-1.95 wherein the pain is associated with allodynia and/or hyperalgesia;
1.97 Method 1 or any of Methods 1.1-1.96, wherein the patient also suffers from anxiety (including general anxiety, social anxiety, and panic disorders), depression (for example refractory depression and MDD), psychosis (including psychosis associated with dementia, such as hallucinations in advanced Parkinson's disease or paranoid delusions), schizophrenia, migraine, substance abuse disorder, substance use disorder, opiate use disorder, or other drug dependencies, for example, stimulant dependency and/or alcohol dependency.
1.98 Method 1 or any of 1.1-1.97, wherein the patient has been diagnosed with a substance use disorder or a substance abuse disorder, such as opiate use disorder (OUD);
1.99 Method 1 or any of Methods 1.1-1.98, wherein said patient has a history of prior substance use or substance abuse with an opiate or opioid drug, e.g., morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentanyl, alfentanyl, trefantinil, brifentanil, remifentanil, octfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, and anileridine, or any combinations thereof;
1.100 Method 1 or any of 1.1-1.99, wherein said patient is or has been diagnosed with an opiate dependency, cocaine dependency, amphetamine dependency, and/or alcohol dependency, or suffers from withdrawal from drug or alcohol dependency (e.g. opiate, cocaine, or amphetamine dependency);
1.101 Method 1 or any of 1.1-1.100, wherein said patient has previously suffered from an opiate overdose;
1.102 Method 1 or any of 1.1-1.100, wherein said the method comprising administering to the patient an effective amount of the Compound of Formula I;
1.103 Method 1.98, wherein the effective amount is 1 mg-1000 mg, for example 2.5 mg-50 mg, or for a long-acting formulation, 25 mg-1500 mg, for example, 50 mg to 500 mg, or 250 mg to 1000 mg, or 250 mg to 750 mg, or 75 mg to 300 mg;
1.104 Method 1.103, wherein the effective amount is 1 mg-100 mg per day, for example 2.5 mg-60 mg per day, or 2.5 mg to 45 mg per day, or 5 mg to 25 mg per day;
1.105 Any foregoing method, wherein the method further comprises the concurrent administration of a selective serotonin reuptake inhibitors (SSRI), e.g., administered simultaneously, separately or sequentially;
1.106 Method 1.105, wherein the SSRI is selected from citalopram, escitalopram, fluoxetine, fluvoxamine, paroxetine, and sertraline
1.107 Any foregoing method, wherein the method further comprises the concurrent administration of a serotonin-norepinephrine reuptake inhibitors (SNRI), e.g., administered simultaneously, separately or sequentially;
1.108 Method 1.107, wherein the SNRI is selected from venlafaxine, sibutramine, duloxetine, atomoxetine, desvenlafaxine, milnacipran, and levomilnacipran;
1.109 Any foregoing method, wherein the method further comprises the concurrent administration of an antipsychotic agent, e.g., administered simultaneously, separately or sequentially;
1.110 Method 1.109, wherein the antipsychotic agent is selected from clomipramine, chlorpromazine, haloperidol, droperidol, fluphenazine, loxapine, mesoridazine, molindone, perphenazine, pimozide, prochlorperazine, promazine, thioridazine, thiothixene, trifluoperazine, brexpiprazole, cariprazine, asenapine, lurasidone, clozapine, aripiprazole, olanzapine, quetiapine, risperidone, ziprasidone and paliperidone;
1.111 Any foregoing method, wherein the method further comprises the concurrent administration of a NMDA receptor antagonist, e.g., administered simultaneously, separately or sequentially;
1.112 Method 1.111, wherein the NMDA receptor antagonist is selected from the group consisting of ketamine (e.g., S-ketamine and/or R-ketamine), hydroxynorketamine, memantine, dextromethorphan, dextroallorphan, dextrorphan, amantadine, and agmatine, or any combination thereof;
1.113 Any foregoing method, wherein the method further comprises the concurrent administration of a compound that modulates GABA activity (e.g., enhances the activity and facilitates GABA transmission), e.g., administered simultaneously, separately or sequentially;
1.114 Method 1.113, wherein the GABA modulating compound is selected from a group consisting of one or more of doxepin, alprazolam, bromazepam, clobazam, clonazepam, clorazepate, diazepam, flunitrazepam, flurazepam, lorazepam, midazolam, nitrazepam, oxazepam, temazepam, triazolam, indiplon, zopiclone, eszopiclone, zaleplon, Zolpidem, gaboxadol, vigabatrin, tiagabine, EVT 201 (Evotec Pharmaceuticals) and estazolam;
1.115 Any foregoing method, wherein the method further comprises the concurrent administration of a 5-HT_{2A} receptor antagonist, e.g., administered simultaneously, separately or sequentially;
1.116 Method 1.115, wherein said additional 5-HT_{2A} receptor antagonist is selected from one or more of pimavanserin, ketanserin, risperidone, eplivanserin, volinanserin (Sanofi-Aventis, France), pruvanserin, MDL 100907 (Sanofi-Aventis, France), HY 10275 (Eli Lilly), APD 125 (Arena Pharmaceuticals, San Diego, CA), and AVE8488 (Sanofi-Aventis, France);
1.117 Any foregoing method, wherein the method further comprises the concurrent administration of a serotonin receptor antagonist/reuptake inhibitor (SARI), e.g., administered simultaneously, separately or sequentially;
1.118 Method 1.117, wherein the serotonin receptor antagonist/reuptake inhibitor (SARI) is selected from a group consisting of one or more ritanserin, nefazodone, serzone and trazodone;
1.119 Any foregoing method, wherein the method further comprises the concurrent administration of an anti-depressant, e.g., administered simultaneously, separately or sequentially;
1.120 Method 1.119, wherein the anti-depressant is selected from amitriptyline, amoxapine, bupropion, citalopram, clomipramine, desipramine, doxepin, duloxetine, escitalopram, fluoxetine, fluvoxamine, imipramine, isocarboxazid, maprotiline, mirtazapine, nefazodone, nortriptyline, paroxetine, phenelzine sulfate, protriptyline, sertraline, tranylcypromine, trazodone, trimipramine, and venlafaxine;
1.121 Any foregoing method, wherein the method further comprises the concurrent administration of an opiate agonist or partial opiate agonist, e.g., administered simultaneously, separately or sequentially;
1.122 Method 1.121, wherein the opiate agonist or partial opiate agonist is a mu-agonist or partial agonist, or a kappa-agonist or partial agonist, including mixed agonist/antagonists (e.g., an agent with partial mu-agonist activity and kappa-antagonist activity);
1.123 Method 1.122, wherein the opiate agonist or partial agonist is buprenorphine, optionally, wherein said method does not include co-treatment with an anxiolytic agent, e.g., a GABA compound or benzodiazepine;
1.124 Any foregoing method, wherein the method further comprises the concurrent administration of an opiate receptor antagonist or inverse agonist, e.g., administered simultaneously, separately or sequentially;
1.125 Method 1.124, wherein the opiate receptor antagonist or inverse agonist is a full opiate antagonist, e.g., selected from naloxone, naltrexone, nalmefene, methadone, nalorphine, levallorphan, samidorphan, nalodeine, cyprodime, or norbinaltorphimine.
1.126 Method 1 or any of 1.1-1.125, wherein the patient was previously treated with another pain-relieving medication, and the patient did not respond adequately to said medication, e.g., the patient's pain did not abate sufficiently, or the patient suffered from side-effects which precluded continued treatment.
1.127 Method 1.126, wherein the patient developed or became at risk of developing an addiction to said other pain-relieving medication.
1.128 Method 1.126 or 1.127, wherein said other pain-relieving medication is selected from non-opiate analgesics (e.g., non-steroidal anti-inflammatory medications, such as ibuprofen, naproxen, ketoprofen, flurbiprofen, fenoprofen, oxaprozin, meclofenamate, mefenamic acid, phenylbutazone, indomethacin, ketorolac, diclofenac, sulindac, etodolac, tolmetin, nabumetone, piroxicam, acetaminophen, aspirin, celecoxib, rofecoxib, valdecoxib, parecoxib, lumiracoxib, etoricoxib, firocoxib), opiate analgesics (e.g., morphine, codeine, oxycodone, hydrocodone, hydromorphone, oxymorphone, buprenorphine, fentanyl, levorphanol, meperidine, nalbuphine, pentazocine, tramadol, methadone), and topical anesthetics (e.g., benzocaine, lidocaine, procaine, bupivacaine, tetracaine) or other medications (e.g., tricyclic antidepressants or anticonvulsants, such as amitriptyline, desipramine, duloxetine, pregabalin, gabapentin, valproate, carbamazepine, phenytoin).

In another embodiment, the present disclosure provides any of Methods 1.1-1.128, wherein the Compound of the present disclosure, or pharmaceutical composition comprising it, is administered for controlled-and/or sustained-release of the Compounds over a period of from about 14 days, about 30 to about 180 days, preferably over the period of about 30, about 60 or about 90 days. Controlled-and/or sustained-release is particularly useful for circumventing premature discontinuation of therapy, particularly for antipsychotic drug therapy where non-compliance or non-adherence to medication regimes is a common occurrence.

In some embodiments, the pain is caused by post-herpetic neuralgia. Postherpetic neuralgia (PHN) is neuropathic pain which occurs due to damage to a peripheral nerve caused by the reactivation of the varicella zoster virus.

In some embodiments, the pain is caused by fibromyalgia, e.g., the pain is a symptom of fibromyalgia. Fibromyalgia is a complex syndrome of uncertain cause or origin. It is classified as a disorder of pain processing, and in particular, the processing of pain signals within the central nervous system. As such, it is like a central neuropathic pain syndrome, and it is often considered an example of “central sensitization.” Fibromyalgia is marked by chronic, widespread pain, often including allodynia. In the United States, only pregabalin and duloxetine have been approved for managing fibromyalgia, and existing analgesics have generally been ineffective.

Patients who suffer from a neuropathy who might otherwise be treated with an opioid analgesic, or other drugs associated with high risk of abuse, would be contra-indicated for such treatment if they suffer from a substance-use disorder or substance abuse disorder, or have had prior instances of opioid addiction, opioid withdrawal, or opioid overdose, or prior instances of substance abuse or alcohol abuse. Therefore, especially in such patients, there is a need for alternative, non-addictive treatment methods, such as the methods described herein.

Substance-use disorders and substance-induced disorders are the two categories of substance-related disorders defined by the Fifth Edition of the DSM (the Diagnostic and Statistical Manual of Mental Disorders, DSM-5). A substance-use disorder is a pattern of symptoms resulting from use of a substance which the individual continues to take, despite experiencing problems as a result. A substance-induced disorder is a disorder induced by use if the substance. Substance-induced disorders include intoxication, withdrawal, substance induced mental disorders, including substance induced psychosis, substance induced bipolar and related disorders, substance induced depressive disorders, substance induced anxiety disorders, substance induced obsessive-compulsive and related disorders, substance induced sleep disorders, substance induced sexual dysfunctions, substance induced delirium and substance induced neurocognitive disorders.

The DSM-5 includes criteria for classifying a substance use disorder as mild, moderate or severe. In some embodiments of the methods disclosed herein, the substance use disorder is selected from a mild substance use disorder, a moderate substance use disorder or a severe substance use disorder. In some embodiments, the substance use disorder is a mild substance use disorder. In some embodiments, the substance use disorder is a moderate substance use disorder. In some embodiments, the substance use disorder is a severe substance use disorder.

Anxiety and depression are highly prevalent co-morbid disorders in patients undergoing treatment of substance use or substance abuse. A common treatment for substance abuse disorder is the combination of the partial opioid agonist buprenorphine with the opioid antagonist naloxone, but neither of these drugs has any significant effect on anxiety or depression, thus leading to the common result that a third drug, such as a benzodiazepine-class anxiolytic agent or an SSRI anti-depressant, must also be prescribed. This makes treatment regimens and patient compliance more difficult. In contrast, the Compounds of the present disclosure provide opiate antagonism along with serotonin antagonism and dopamine modulation. This may result in significant enhancement of treatment of patients with substance use or abuse disorder concomitant with anxiety and/or depression.

The compounds of the present disclosure may have anxiolytic properties ameliorating the need for treatment of a patient with an anxiolytic agent where said patients suffers from co-morbid anxiety. Thus, in some embodiments, the present disclosure provides a method according to Method 1 et seq., wherein patient suffers from anxiety or symptoms of anxiety or who is diagnosed with anxiety as a co-morbid disorder, or as a residual disorder, wherein the method does not comprise the further administration of an anxiolytic agent, such as a benzodiazepine and other described herein.

In any of the embodiments of Method 1 et seq. wherein the Compound of the present disclosure is administered along with one or more second therapeutic agents, the one or more second therapeutic agents may be administered as a part of the pharmaceutical composition comprising the Compound of the present disclosure. Alternatively, the one or more second therapeutic agents may be administered in separate pharmaceutical compositions (such as pills, tablets, capsules and injections) administered simultaneously, sequentially or separately from the administration of the Compound of the present disclosure.

In a second aspect, the present disclosure provides use of a Compound of the present disclosure, e.g., a Compound of Formula I or any of the compounds described in any of the embodiments of Methods 1.1 to 1.71, in the manufacture of a medicament for use according to Method 1 or any of Methods 1.1-1.128.

In a third aspect, the present disclosure provides a Compound of the present disclosure, e.g., a Compound of Formula I or any of the compounds described in any of the embodiments of Methods 1.1 to 1.71, for use according to Method 1 or any of Methods 1.1-1.128.

Without being bound by theory, it is believed that the Compounds of the present disclosure, such as the Compound of Formula A, are potent 5-HT_{2A}, D₁ and Mu opiate modulators (e.g., antagonists), which also provide moderate D₂ and SERT modulation (e.g., antagonism). Furthermore, it has been unexpectedly found that such compounds may operate as “biased” Mu opiate ligands. This means that when the compounds bind to Mu opiate receptors, they may operate as partial Mu agonists via G-protein coupled signaling, but as Mu antagonists via beta-arrestin signaling. This is in contrast to traditional opiate agonists, such as morphine and fentanyl, which tend to strongly activate both G-protein signaling and beta-arrestin signaling. The activation of beta-arrestin signaling by such drugs is thought to mediate the gastrointestinal dysfunction and respiratory suppression typically mediated by opiate drugs. As a result, Compounds of the present disclosure, e.g., Compounds of Formula I, are therefore expected to result in pain amelioration with less severe gastrointestinal and respiratory side effects than existing opiate analgesics. This effect has been shown in pre-clinical studies and Phase II and Phase III clinical trials of the biased Mu agonist oliceridine. Oliceridine has been shown to result in biased mu agonism via G-protein coupled signaling with reduced beta-arresting signaling compared to morphine, and this has been linked to its ability to produce analgesia with reduced respiratory side effects compared to morphine. Furthermore, because these compounds antagonize the beta-arrestin pathway, they are expected to be useful in treating opiate overdose, because they will inhibit the most severe opiate adverse effects while still providing pain relief. Furthermore, these compounds also have sleep maintenance effect due to their serotonergic activity. As many people suffering from chronic pain have difficulty sleeping due to the pain, these compounds can help such patients sleep through the night due to the synergistic effects of serotonergic and opiate receptor activities.

Thus, the Compounds of the present disclosure are effective in treating and/or preventing neuropathic pain in patients having opiate use disorder (OUD), opiate overdose, or opiate withdrawal, either alone, or in conjunction with an opiate antagonist or inverse agonist (e.g., naloxone or naltrexone). Compounds of the present disclosure are expected to show provide potent analgesia but without the adverse effects (e.g., GI effects and pulmonary depression) and abuse potential seen with other opioid treatments (e.g., oxycodone, methadone or buprenorphine). The unique pharmacologic profile of these compounds should also mitigate the risks of adverse drug- drug interactions (e.g., alcohol). These compounds are therefore particularly suited to long-term treatment and maintenance of pain in patients who cannot receive opioid or opiate drugs.

In some embodiments of the present disclosure, the compounds of Formula I have one or more biologically labile functional groups positioned within the compounds such that natural metabolic activity will remove the labile functional groups, resulting in another Compound of Formula I. For example, when group R¹ is C(O)—O—C(R^{a})(R^{b})(R^{c}), —C(O)—O—CH₂—O—C(R^{a})(R^{b})(R^{c}) or —C(R⁶)(R⁷)—O—C(O)—R⁸, under biological conditions this substituent will undergo hydrolysis to yield the same compound wherein R¹ is H, thus making the original compounds prodrugs of the compound wherein R¹ is H. Some of such prodrug compounds may have little-to-no or only moderate biological activity but upon hydrolysis to the compound wherein R¹ is H, the compound may have strong biological activity. As such, depending on the compound selected, administration of the compounds of the present disclosure to a patient in need thereof may result in immediate biological and therapeutic effect, or immediate and delayed biological and therapeutic effect, or only delayed biological and therapeutic effect. Such prodrug compounds will thus serve as a reservoir of the pharmacologically active compounds of Formula I wherein R¹ is H. In this way, some compounds of the present disclosure are particularly suited to formulation as long-acting injectable (LAI) or “Depot” pharmaceutical compositions. Without being bound by theory, an injected “depot” comprising a compound of the present disclosure will gradually release into the body tissues said compound, in which tissues said compound will be gradually metabolized to yield a compound of Formula I wherein R¹ is H. Such depot formulations may be further adjusted by the selection of appropriate components to control the rate of dissolution and release of the compounds of the present disclosure. Such prodrug forms of compounds related to the Compounds of Formula I have previously been disclosed, e.g., in WO 2019/23063.

“Alkyl” as used herein is a saturated or unsaturated hydrocarbon moiety, e.g., one to twenty-one carbon atoms in length, unless indicated otherwise; any such alkyl may be linear or branched (e.g., n-butyl or tert-butyl), preferably linear, unless otherwise specified. For example, “C₁₋₂₁ alkyl” denotes alkyl having 1 to 21 carbon atoms. In one embodiment, alkyl is optionally substituted with one or more hydroxy or C₁₋₂₂alkoxy (e.g., ethoxy) groups. In another embodiment, alkyl contains 1 to 21 carbon atoms, preferably straight chain and optionally saturated or unsaturated, for example in some embodiments wherein R¹ is an alkyl chain containing 1 to 21 carbon atoms, preferably 6-15 carbon atoms, 16-21 carbon atoms, e.g., so that together with the —C(O)— to which it attaches, e.g., when cleaved from the compound of Formula I, forms the residue of a natural or unnatural, saturated or unsaturated fatty acid.

The term “pharmaceutically acceptable diluent or carrier” is intended to mean diluents and carriers that are useful in pharmaceutical preparations, and that are free of substances that are allergenic, pyrogenic or pathogenic, and that are known to potentially cause or promote illness. Pharmaceutically acceptable diluents or carriers thus exclude bodily fluids such as example blood, urine, spinal fluid, saliva, and the like, as well as their constituent components such as blood cells and circulating proteins. Suitable pharmaceutically acceptable diluents and carriers can be found in any of several well-known treatises on pharmaceutical formulations, for example Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; and Martindale, The Extra Pharmacopoeia, Thirty-Second Edition (The Pharmaceutical Press, London, 1999); all of which are incorporated by reference herein in their entirety.

The terms “purified,” “in purified form” or “in isolated and purified form” for a compound refers to the physical state of said compound after being isolated from a synthetic process (e.g., from a reaction mixture), or natural source or combination thereof. Thus, the term “purified,” “in purified form” or “in isolated and purified form” for a compound refers to the physical state of said compound after being obtained from a purification process or processes described herein or well known to the skilled artisan (e.g., chromatography, recrystallization, LC-MS and LC-MS/MS techniques and the like), in sufficient purity to be characterizable by standard analytical techniques described herein or well known to the skilled artisan.

Unless otherwise indicated, the Compounds of the present disclosure may exist in free base form or in salt form, such as a pharmaceutically acceptable salt form, e.g., as acid addition salts. An acid-addition salt of a compound of the present disclosure which is sufficiently basic, for example, an acid-addition salt with, for example, an inorganic or organic acid, for example hydrochloric acid or toluenesulfonic acid. In addition, a salt of a compound of the present disclosure which is sufficiently acidic is an alkali metal salt, for example a sodium or potassium salt, or a salt with an organic base which affords a physiologically-acceptable cation. In a particular embodiment, the salt of the Compounds of the present disclosure is a toluenesulfonic acid addition salt.

The Compounds of the present disclosure are intended for use as pharmaceuticals, therefore pharmaceutically acceptable salts are preferred. Salts which are unsuitable for pharmaceutical uses may be useful, for example, for the isolation or purification of free Compounds of the present disclosure, and are therefore also included within the scope of the compounds of the present disclosure.

The Compounds of the present disclosure may comprise one or more chiral carbon

atoms. The compounds thus exist in individual isomeric, e.g., enantiomeric or diastereomeric form or as mixtures of individual forms, e.g., racemic/diastereomeric mixtures. Any isomer may be present in which the asymmetric center is in the (R)-, (S)-, or (R,S)-configuration. The invention is to be understood as embracing both individual optically active isomers as well as mixtures (e.g., racemic/diastereomeric mixtures) thereof. Accordingly, the Compounds of the present disclosure may be a racemic mixture or it may be predominantly, e.g., in pure, or substantially pure, isomeric form, e.g., greater than 70% enantiomeric/diastereomeric excess (“ee”), preferably greater than 80% ee, more preferably greater than 90% ee, most preferably greater than 95% ee. The purification of said isomers and the separation of said isomeric mixtures may be accomplished by standard techniques known in the art (e.g., column chromatography, preparative TLC, preparative HPLC, simulated moving bed and the like).

Geometric isomers by nature of substituents about a double bond or a ring may be present in cis (Z) or trans (E) form, and both isomeric forms are encompassed within the scope of this invention.

It is also intended that the compounds of the present disclosure encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the compounds of the disclosure may be replaced with deuterium (a stable isotope which is non-radioactive). Examples of known stable isotopes include, but not limited to, deuterium (²H or D), ¹³C, ¹⁵N, ¹⁸O. Alternatively, unstable isotopes, which are radioactive isotopes which contain additional neutrons compared to the abundant nuclides of the same species (i.e., element), e.g., ¹²³I, ¹³¹I, ¹²⁵I, ¹¹C, ¹⁸F, may replace the corresponding abundant species of I, C and F. Another example of useful isotope of the compound of the present disclosure is the ¹¹C isotope. These radio isotopes are useful for radio-imaging and/or pharmacokinetic studies of the compounds of the present disclosure. In addition, the substitution of atoms of having the natural isotopic distributing with heavier isotopes can result in desirable change in pharmacokinetic rates when these substitutions are made at metabolically liable sites. For example, the incorporation of deuterium (²H) in place of hydrogen can slow metabolic degradation when the position of the hydrogen is a site of enzymatic or metabolic activity.

Compounds of the present disclosure may be administered in the form of a pharmaceutical composition which is a depot formulation, e.g., by dispersing, dissolving, suspending or encapsulating the Compounds of the present disclosure in a polymeric matrix as described hereinbefore, such that the Compound is continually released as the polymer degrades over time. The release of the Compounds of the present disclosure from the polymeric matrix provides for the controlled-and/or delayed-and/or sustained-release of the Compounds, e.g., from the pharmaceutical depot composition, into a subject, for example a warm-blooded animal such as man, to which the pharmaceutical depot is administered. Thus, the pharmaceutical depot delivers the Compounds of the present disclosure to the subject at concentrations effective for treatment of the particular disease or medical condition over a sustained period of time, e.g., 14-180 days, preferably about 30, about 60 or about 90 days.

Polymers useful for the polymeric matrix in the Composition of the present disclosure (e.g., Depot composition of the present disclosure) may include a polyester of a hydroxyfatty acid and derivatives thereof or other agents such as polylactic acid, polyglycolic acid, polycitric acid, polymalic acid, poly-beta.-hydroxybutyric acid, epsilon.-capro-lactone ring opening polymer, lactic acid-glycolic acid copolymer, 2-hydroxybutyric acid-glycolic acid copolymer, polylactic acid-polyethylene glycol copolymer or polyglycolic acid-polyethylene glycol copolymer), a polymer of an alkyl alpha-cyanoacrylate (for example poly(butyl 2-cyanoacrylate)), a polyalkylene oxalate (for example polytrimethylene oxalate or polytetramethylene oxalate), a polyortho ester, a polycarbonate (for example polyethylene carbonate or polyethylenepropylene carbonate), a polyortho-carbonate, a polyamino acid (for example poly-gamma.-L-alanine, poly-. gamma.-benzyl-L-glutamic acid or poly-y-methyl-L-glutamic acid), a hyaluronic acid ester, and the like, and one or more of these polymers can be used.

If the polymers are copolymers, they may be any of random, block and/or graft copolymers. When the above alpha-hydroxycarboxylic acids, hydroxydicarboxylic acids and hydroxytricarboxylic acids have optical activity in their molecules, any one of D-isomers, L-isomers and/or DL-isomers may be used. Among others, alpha-hydroxycarboxylic acid polymer (preferably lactic acid-glycolic acid polymer), its ester, poly-alpha-cyanoacrylic acid esters, etc. may be used, and lactic acid-glycolic acid copolymer (also referred to as poly(lactide-alpha-glycolide) or poly(lactic-co-glycolic acid), and hereinafter referred to as PLGA) are preferred. Thus, in one aspect the polymer useful for the polymeric matrix is PLGA. As used herein, the term PLGA includes polymers of lactic acid (also referred to as polylactide, poly (lactic acid), or PLA). Most preferably, the polymer is the biodegradable poly (d,1-lactide-co-glycolide) polymer.

In a preferred embodiment, the polymeric matrix of the present disclosure is a biocompatible and biodegradable polymeric material. The term “biocompatible” is defined as a polymeric material that is not toxic, is not carcinogenic, and does not significantly induce inflammation in body tissues. The matrix material should be biodegradable wherein the polymeric material should degrade by bodily processes to products readily disposable by the body and should not accumulate in the body. The products of the biodegradation should also be biocompatible with the body in that the polymeric matrix is biocompatible with the body. Particular useful examples of polymeric matrix materials include poly(glycolic acid), poly-D,L-lactic acid, poly-L-lactic acid, copolymers of the foregoing, poly(aliphatic carboxylic acids), copolyoxalates, polycaprolactone, polydioxanone, poly(ortho carbonates), poly (acetals), poly(lactic acid-caprolactone), polyortho esters, poly(glycolic acid-caprolactone), polyanhydrides, and natural polymers including albumin, casein, and waxes, such as, glycerol mono-and distearate, and the like. The preferred polymer for use in the practice of this invention is dl(polylactide-co-glycolide). It is preferred that the molar ratio of lactide to glycolide in such a copolymer be in the range of from about 75:25 to 50:50.

Useful PLGA polymers may have a weight-average molecular weight of from about 5,000 to 500,000 Daltons, preferably about 150,000 Daltons. Dependent on the rate of degradation to be achieved, different molecular weight of polymers may be used. For a diffusional mechanism of drug release, the polymer should remain intact until all of the drug is released from the polymeric matrix and then degrade. The drug can also be released from the polymeric matrix as the polymeric excipient bioerodes.

The PLGA may be prepared by any conventional method, or may be commercially available. For example, PLGA can be produced by ring-opening polymerization with a suitable catalyst from cyclic lactide, glycolide, etc. (see EP-0058481B2; Effects of polymerization variables on PLGA properties: molecular weight, composition and chain structure).

It is believed that PLGA is biodegradable by means of the degradation of the entire solid polymer composition, due to the break-down of hydrolysable and enzymatically cleavable ester linkages under biological conditions (for example in the presence of water and biological enzymes found in tissues of warm-blooded animals such as humans) to form lactic acid and glycolic acid. Both lactic acid and glycolic acid are water-soluble, non-toxic products of normal metabolism, which may further biodegrade to form carbon dioxide and water. In other words, PLGA is believed to degrade by means of hydrolysis of its ester groups in the presence of water, for example in the body of a warm-blooded animal such as man, to produce lactic acid and glycolic acid and create the acidic microclimate. Lactic and glycolic acid are by-products of various metabolic pathways in the body of a warm-blooded animal such as man under normal physiological conditions and therefore are well tolerated and produce minimal systemic toxicity.

In another embodiment, the polymeric matrix may comprise a star polymer wherein the structure of the polyester is star-shaped. These polyesters have a single polyol residue as a central moiety surrounded by acid residue chains. The polyol moiety may be, e. g., glucose or, e. g., mannitol. These esters are known and described in GB 2,145,422 and in U.S. Pat. No. 5,538,739, the contents of which are incorporated by reference.

The star polymers may be prepared using polyhydroxy compounds, e. g., polyol, e.g., glucose or mannitol as the initiator. The polyol contains at least 3 hydroxy groups and has a molecular weight of up to about 20,000 Daltons, with at least 1, preferably at least 2, e.g., as a mean 3 of the hydroxy groups of the polyol being in the form of ester groups, which contain polylactide or co-polylactide chains. The branched polyesters, e.g., poly (d, 1-lactide-co-glycolide) have a central glucose moiety having rays of linear polylactide chains.

The depot compositions of the present disclosure (e.g., Pharmaceutical Compositions I-A or I-B), in a polymer matrix) as hereinbefore described may comprise the polymer in the form of microparticles or nanoparticles, or in a liquid form, with the Compounds of the present disclosure dispersed or encapsulated therein. “Microparticles” is meant solid particles that contain the Compounds of the present disclosure either in solution or in solid form wherein such compound is dispersed or dissolved within the polymer that serves as the matrix of the particle. By an appropriate selection of polymeric materials, a microparticle formulation can be made in which the resulting microparticles exhibit both diffusional release and biodegradation release properties.

When the polymer is in the form of microparticles, the microparticles may be prepared using any appropriate method, such as by a solvent evaporation or solvent extraction method. For example, in the solvent evaporation method, the Compounds of the present disclosure and the polymer may be dissolved in a volatile organic solvent (for example a ketone such as acetone, a halogenated hydrocarbon such as chloroform or methylene chloride, a halogenated aromatic hydrocarbon, a cyclic ether such as dioxane, an ester such as ethyl acetate, a nitrile such as acetonitrile, or an alcohol such as ethanol) and dispersed in an aqueous phase containing a suitable emulsion stabilizer (for example polyvinyl alcohol, PVA). The organic solvent is then evaporated to provide microparticles with the Compounds of the present disclosure encapsulated therein. In the solvent extraction method, the Compounds of the present disclosure and polymer may be dissolved in a polar solvent (such as acetonitrile, dichloromethane, methanol, ethyl acetate or methyl formate) and then dispersed in an aqueous phase (such as a water/PVA solution). An emulsion is produced to provide microparticles with the Compounds of the present disclosure encapsulated therein. Spray drying is an alternative manufacturing technique for preparing the microparticles.

Another method for preparing the microparticles of the present disclosure is also described in both U.S. Pat. Nos. 4,389,330 and 4,530,840.

The microparticle can be prepared by any method capable of producing microparticles in a size range acceptable for use in an injectable composition. One preferred method of preparation is that described in U.S. Pat. No. 4,389,330. In this method the active agent is dissolved or dispersed in an appropriate solvent. To the agent-containing medium is added the polymeric matrix material in an amount relative to the active ingredient that provides a product having the desired loading of active agent. Optionally, all of the ingredients of the microparticle product can be blended in the solvent medium together.

Solvents for the Compounds of the present disclosure and the polymeric matrix material that can be employed in the practice of the present invention include organic solvents, such as acetone; halogenated hydrocarbons, such as chloroform, methylene chloride, and the like; aromatic hydrocarbon compounds; halogenated aromatic hydrocarbon compounds; cyclic ethers; alcohols, such as, benzyl alcohol; ethyl acetate; and the like. In one embodiment, the solvent for use in the practice of the present invention may be a mixture of benzyl alcohol and ethyl acetate. Further information for the preparation of microparticles useful for the invention can be found in U.S. Patent Publication Number 2008/0069885, the contents of which are incorporated herein by reference in their entirety.

The amount of the Compounds of the present disclosure incorporated in the microparticles usually ranges from about 1 wt % to about 90 wt. %, preferably 30 to 50 wt. %, more preferably 35 to 40 wt. %. By weight % is meant parts of the Compounds of the present disclosure per total weight of microparticle.

The pharmaceutical depot compositions may comprise a pharmaceutically-acceptable diluent or carrier, such as a water miscible diluent or carrier.

Details of Osmotic-controlled Release Oral Delivery System composition may be found in EP 1 539 115 (U.S. Pub. No. 2009/0202631) and WO 2000/35419 (US 2001/0036472), the contents of each of which are incorporated by reference in their entirety.

An “effective amount” means a “therapeutically effective amount”, that is, any amount of the Compounds of the present disclosure (for example as contained in the pharmaceutical composition or dosage form) which, when administered to a subject suffering from a disease or disorder, is effective to cause a reduction, remission, or regression of the disease or disorder over the period of time as intended for the treatment.

Dosages employed in practicing the present invention will of course vary depending, e.g. on the particular disease or condition to be treated, the particular Compound of the present disclosure used, the mode of administration, and the therapy desired. Unless otherwise indicated, an amount of the Compound of the present disclosure for administration (whether administered as a free base or as a salt form) refers to or is based on the amount of the Compound of the present disclosure in free base form (i.e., the calculation of the amount is based on the free base amount).

Compounds of the present disclosure may be administered by any satisfactory route, including orally, parenterally (intravenously, intramuscular or subcutaneous) or transdermally. In certain embodiments, the Compounds of the present disclosure, e.g., in depot formulation, is preferably administered parenterally, e.g., by injection, for example, intramuscular or subcutaneous injection.

In general, satisfactory results for Method 1 et seq., as set forth above are indicated to be obtained on oral administration at dosages of the order from about 1 mg to 100 mg once daily, preferably 2.5 mg-50 mg, e.g., 2.5 mg, 5 mg, 10 mg, 20 mg, 30 mg, 40 mg or 50 mg, once daily, preferably via oral administration.

For treatment of the disorders disclosed herein wherein the depot composition is used to achieve longer duration of action, the dosages will be higher relative to the shorter action composition, e.g., higher than 1-100 mg, e.g., 25 mg, 50 mg, 100 mg, 500 mg, 1000 mg, or greater than 1000 mg. Duration of action of the Compounds of the present disclosure may be controlled by manipulation of the polymer composition, i.e., the polymer: drug ratio and microparticle size. Wherein the composition of the present disclosure is a depot composition, administration by injection is preferred.

The pharmaceutically acceptable salts of the Compounds of the present disclosure can be synthesized from the parent compound which contains a basic or acidic moiety by conventional chemical methods. Generally, such salts can be prepared by reacting the free base forms of these compounds with a stoichiometric amount of the appropriate acid in water or in an organic solvent, or in a mixture of the two; generally, non-aqueous media like ether, ethyl acetate, ethanol, isopropanol, or acetonitrile are preferred. Further details for the preparation of these salts, e.g., toluenesulfonic salt in amorphous or crystal form, may be found in US 2011/112105.

Pharmaceutical compositions comprising Compounds of the present disclosure may be prepared using conventional diluents or excipients (an example include, but is not limited to sesame oil) and techniques known in the galenic art. Thus, oral dosage forms may include tablets, capsules, solutions, suspensions and the like.

The term “concurrently” when referring to a therapeutic use means administration of two or more active ingredients to a patient as part of a regimen for the treatment of a disease or disorder, whether the two or more active agents are given at the same or different times or whether given by the same or different routes of administrations. Concurrent administration of the two or more active ingredients may be at different times on the same day, or on different dates or at different frequencies.

The term “simultaneously” when referring to a therapeutic use means administration of two or more active ingredients at or about the same time by the same route of administration.

The term “separately” when referring to a therapeutic use means administration of two or more active ingredients at or about the same time by different route of administration.

### Methods of Making the Compounds of the Present Disclosure

The Compound of Formula A, and methods for its synthesis, including the synthesis of intermediates used in the synthetic schemes described below, have been disclosed in, for example, U.S. Pat. No. 8,309,722, and US 2017/319580. The synthesis of similar fused gamma-carbolines has been disclosed in, for example, U.S. Pat. No. 8,309,722, U.S. Pat. No. 8,993,572, US 2017/0183350, WO 2018/126140 and WO 2018/126143, the contents of each of which are incorporated by reference in their entireties. Compounds of the present disclosure can be prepared using similar procedures.

Compounds of Formula I wherein R¹ is C(O)—O—C(R^{a})(R^{b})(R^{c}), —C(O)—O—CH₂—O—C(R^{a})(R^{b})(R^{c}) or —C(R⁶)(R⁷)—O—C(O)—R⁸, may be prepared according to the procedures disclosed in international application PCT/US2018/043102.

Other Compounds of the present disclosure came be made by analogous procedures known to those skilled in the art.

Isolation or purification of the diastereomers of the Compounds of the present disclosure may be achieved by conventional methods known in the art, e.g., column purification, preparative thin layer chromatography, preparative HPLC, crystallization, trituration, simulated moving beds and the like.

Salts of the Compounds of the present disclosure may be prepared as similarly described in U.S. Pat. Nos. 6,548,493; 7,238,690; 6,552,017; 6,713,471; 7,183,282, 8,648,077; 9,199,995; 9,586,860; U.S. RE39680; and U.S. RE39679, the contents of each of which are incorporated by reference in their entirety.

Diastereomers of prepared compounds can be separated by, for example, HPLC using CHIRALPAK® AY-H, 5 μ, 30×250 mm at room temperature and eluted with 10% ethanol/90% hexane/0.1% dimethylethylamine. Peaks can be detected at 230 nm to produce 98-99.9% ee of the diastereomer.

### EXAMPLES

### Example 1: Synthesis of (6bR,10aS)-8-(3-(4-fluorophenoxy)propyl)-6b,7,8,9,10,10a-hexahydro-1H-pyrido [3′,4′:4,5]pyrrolo[1,2,3-de]quinoxalin-2(3H)-one

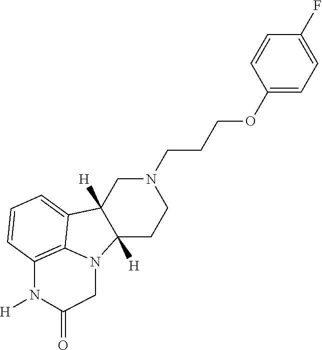 [SMILES: O=C1CN2c3c(cccc3[C@@H]3CN(CCCOc4ccc(F)cc4)CC[C@@H]32)N1]

A mixture of (6bR,10aS)-66,7,8,9,10,10a-hexahydro-1H-pyrido [3′,4′:4,5]pyrrolo[1,2,3-de]quinoxalin-2(3H)-one (100 mg, 0.436 mmol), 1-(3-chloroproxy)-4-fluorobenzene (100 μL, 0.65 mmol) and potassium iodide (KI) (144 mg, 0.87 mmol) in dimethylformamide (DMF) (2 mL) is degassed with argon for 3 minutes and N,N-diisopropylethylamine (DIPEA) (150 μL, 0.87 mmol) is added. The resulting mixture is heated to 78° C. and stirred at this temperature for 2 h. The mixture is cooled to room temperature and then filtered. The filter cake is purified by silica gel column chromatography using a gradient of 0-100% ethyl acetate in a mixture of methanol/7N NH₃ in methanol (1:0.1 v/v) as an eluent to produce partially purified product, which is further purified with a semi-preparative HPLC system using a gradient of 0-60% acetonitrile in water containing 0.1% formic acid over 16 min to obtain the title product as a solid (50 mg, yield 30%). MS (ESI) m/z 406.2 [M+1]⁺. ¹H NMR (500 MHZ, DMSO-d₆) δ10.3 (s, 1H), 7.2-7.1 (m, 2H), 7.0-6.9 (m, 2H), 6.8 (dd, J=1.03, 7.25 Hz, 1H), 6.6 (t, J=7.55 Hz, 1H), 6.6 (dd, J=1.07, 7.79 Hz, 1H), 4.0 (t, J=6.35 Hz, 2H), 3.8 (d, J=14.74 Hz, 1H), 3.3-3.2 (m, 3H), 2.9 (dd, J=6.35, 11.13 Hz, 1H), 2.7-2.6 (m, 1H), 2.5-2.3 (m, 2H), 2.1 (t, J=11.66 Hz, 1H), 2.0 (d, J=14.50 Hz, 1H), 1.9-1.8 (m, 3H), 1.7 (t, J=11.04 Hz, 1H).

### Example 2: Synthesis of (6bR,10aS)-8-(3-(6-fluoro-1H-indazol-3-yl)propyl)-6b,7,8,9,10,10a-hexahydro-1H-pyrido [3′,4′:4,5]pyrrolo[1,2,3-de]quinoxalin-2 (3H)-one

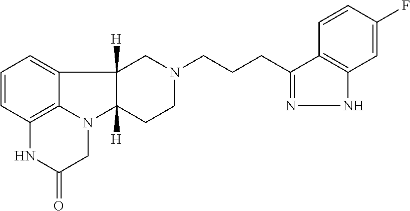 [SMILES: O=C1CN2c3c(cccc3[C@@H]3CN(CCCc4n[nH]c5cc(F)ccc45)CC[C@@H]32)N1]

Step 1: To a stirred solution of BCl₃·MeS (10.8 g, 60 mmol) in toluene at 0-5° C. is added 3-fluoroaniline (5.6 mL, 58 mmol), followed by 4-chlorobutyronitrile (7.12 g. 68.73 mmol) and aluminum chloride (AlCl₃) (8.0 g, 60.01 mmol). The mixture is stirred at 130° C. overnight and cooled to 50° C. Hydrochloric acid (3N, 30 mL) is added carefully and the resulting solution is stirred at 90° C. overnight. The obtained brown solution is cooled to room temperature and evaporated to dryness. The residue is dissolved in dichloromethane (DCM) (20 mL) and basified with saturated Na₂CO₃ to pH=7-8. The organic phase is separated, dried over Na₂CO₃ and then concentrated. The residue is purified by silica-gel column chromatography using a gradient of 0-20% ethyl acetate in hexane as eluent to afford 2′-amino-4-chloro-4′-fluorobutyrophenone as a yellow solid (3.5 g, yield 28%). MS (ESI) m/z 216.1 [M+1]⁺.

Step 2: To a suspension of 2′-amino-4-chloro-4′-fluorobutyrophenone (680 mg, 3.2 mmol) in concentrated HCl (14 mL) at 0-5° C., NaNO₂ (248 mg, 3.5 mmol) in water (3 mL) is added. The resulting brown solution is stirred at 0-5° C. for 1 h and then SnCl₂·2H₂O (1.74 g, 7.7 mmol) in concentrated HCl (3 mL) is added. The mixture is stirred at 0-5° C. for additional 1 hour and then dichloromethane (30 mL) is added. The reaction mixture is filtered and the filtrate is dried over K₂CO₃ and evaporated to dryness. The residue is purified by silica-gel column chromatography using a gradient of 0-35% ethyl acetate in hexane as eluent to yield 3-(3-chloropropyl)-6-fluoro-1H-indazole as a white solid (400 mg, yield 60%). MS (ESI) m/z 213.1 [M+1]⁺.

Step 3: A mixture of (6bR,10aS)-6b,7,8,9,10,10a-hexahydro-1H-pyrido[3′,4′:4,5]pyrrolo[1,2,3-de]quinoxalin-2(3H)-one (100 mg, 0.436 mmol), 3-(3-chloropropyl)-6-fluoro-1/-indazole (124 mg, 0.65 mmol) and KI (144 mg, 0.87 mmol) is degassed with argon for 3 minutes and DIPEA (150 μL, 0.87 mmol) is added. The resulting mixture is stirred at 78° C. for 2 h and then cooled to room temperature. The generated precipitate is filtered. The filter cake is purified with a semi-preparative HPLC system using a gradient of 0-60% acetonitrile in water containing 0.1% formic acid over 16 min to yield (6bR,10aS)-8-(3-(6-fluoro-1H-indazol-3-yl)propyl)-6b,7,8,9,10,10a-hexahydro-1H-pyrido[3′,4′:4,5]pyrrolo[1,2,3-de]quinoxalin-2(3H)-one as an off-white solid (50 mg, yield 28%). MS (ESI) m/z 406.2 [M+1]⁺. ¹H NMR (500 MHZ, DMSO-d₆) δ12.7 (s, 1H), 10.3 (s, 1H), 7.8 (dd, J=5.24, 8.76 Hz, 1H), 7.2 (dd, J=2.19, 9.75 Hz, 1H), 6.9 (ddd, J=2.22, 8.69, 9.41 Hz, 1H), 6.8-6.7 (m, 1H), 6.6 (t, J =7.53 Hz, 1H), 6.6 (dd, J=1.07, 7.83 Hz, 1H), 3.8 (d, J=14.51 Hz, 1H), 3.3-3.2 (m, 1H), 3.2 (s, 2H), 2.9 (dt, J=6.35, 14.79 Hz, 3H), 2.7-2.6 (m, 1H), 2.4-2.2 (m, 2H), 2.1 (t, J=11.42 Hz, 1H), 2.0-1.8 (m, 3H), 1.8-1.7 (m, 1H), 1.7 (t, J=10.89 Hz, 1H).

### Example 3: Synthesis of (6bR,10aS)-8-(3-(6-fluorobenzo[d]isoxazol-3-yl)propyl)-6b,7,8,9,10,10a-hexahydro-1H-pyrido [3′,4′:4,5]pyrrolo[1,2,3-de]quinoxalin-2(3H)-one

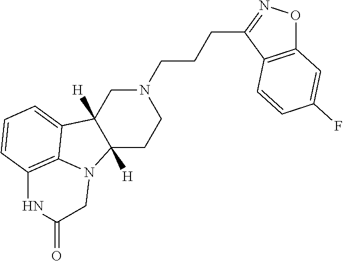 [SMILES: O=C1CN2c3c(cccc3[C@@H]3CN(CCCc4noc5cc(F)ccc45)CC[C@@H]32)N1]

A mixture of (6bR,10aS)-6b,7,8,9,10,10a-hexahydro-1H-pyrido[3′,4′:4,5]pyrrolo[1,2,3-de]quinoxalin-2(3H)-one (148 mg, 0.65 mmol), 3-(3-chloropropyl)-6-fluorobenzo[d]isoxazole (276 mg, 1.3 mmol) and KI (210 mg, 1.3 mmol) is degassed with argon and then DIPEA (220 μL, 1.3 mmol) is added. The resulting mixture is stirred at 78° C. for 2 h and then cooled to room temperature. The mixture is concentrated under vacuum. The residue is suspended in dichloromethane (50 mL) and then washed with water (20 mL). The organic phase is dried over K₂CO₃, filtered, and then concentrated under vacuum. The crude product is purified by silica gel column chromatography with a gradient of 0-10% of methanol in ethyl acetate containing 1% 7N NH₃ to yield the title product as a solid (80 mg, yield 30%). MS (ESI) m/z 407.2 [M+1]⁺. ¹H NMR (500 MHZ, DMSO-d₆) δ10.3 (s, 1H), 8.0-7.9 (m, 1H), 7.7 (dd, J=2.15, 9.19 Hz, 1H), 7.3 (td, J=2.20, 9.09 Hz, 1H), 6.8 (d, J=7.22 Hz, 1H), 6.6 (t, J=7.54 Hz, 1H), 6.6 (d, J=7.75 Hz, 1H), 3.8 (d, J=14.53 Hz, 1H), 3.3 (s, 1H), 3.2 (s, 1H), 3.2-3.1 (m, 1H), 3.0 (t, J=7.45 Hz, 2H), 2.9-2.8 (m, 1H), 2.7-2.5 (m, 1H), 2.4-2.2 (m, 2H), 2.2-2.0 (m, 1H), 2.0-1.8 (m, 3H), 1.8-1.6 (m, 2H).

### Example 4: Synthesis of 4-(3-((6bR,10aS)-2-oxo-2,3,6b,7,10,10a-hexahydro-1H-pyrido[3′,4′:4,5]-pyrrolo[1,2,3-de]quinoxalin-8(9H)-yl) propoxy)benzonitrile

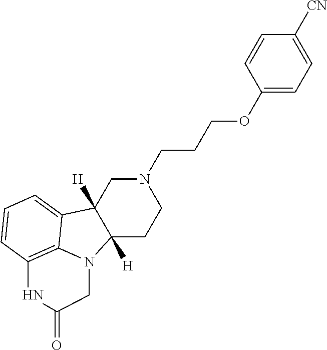 [SMILES: N#Cc1ccc(OCCCN2CC[C@H]3[C@@H](C2)c2cccc4c2N3CC(=O)N4)cc1]

Step 1: A degassed suspension of (4aS,9bR)-ethyl 6-bromo-3,4,4a,5-tetrahydro-1H-pyrido[4,3-b]indole-2(9bH)-carboxylate (21.5 g, 66.2mmol), chloroacetamide (9.3g, 100mmol), and KI (17.7 g, 107mmol) in dioxane (60 mL) is stirred at 104° C. for 48 h. The solvent is removed and the residue is suspended in dichloromethane (200 mL) and extracted with water (100 mL). The separated dichloromethane phase is dried over potassium carbonate (K₂CO₃) for 1 h and then filtered. The filtrate is evaporated to give a crude product as a brown oil. To the brown oil is added ethyl acetate (100 m L) and the mixture is sonicated for 2 min. A yellow solid gradually precipitates from the mixture, which turns into a gel after standing at room temperature for an additional 2 h. Additional ethyl acetate (10 mL) is added and the resulting solid is filtered. The filtered cake is rinsed with ethyl acetate (2 m L) and further dried under high vacuum to produce (4aS,9bR)-ethyl 5-(2-amino-2-oxoethyl)-6-bromo-3,4,4a,5-tetrahydro-1H-pyrido[4,3-b]indole-2(9bH)-carboxylate as an off white solid (19 g, yield 75%). This product is used directly in the next step without further purification. MS (ESI) m/z 382.0 [M+H]⁺.

Step 2: A mixture of (4aS,9bR)-ethyl 5-(2-amino-2-oxoethyl)-6-bromo-3,4,4a,5-tetrahydro-1H-pyrido [4,3-b] indole-2(9bH)-carboxylate (12.9 g, 33.7mmol), KI (10.6g, 63.8mmol), CuI (1.34g, 6.74 mmol) in dioxane (50 mL) is bubbled with argon for 5 min. To this mixture is added N,N,N,N′-tetramethylethylenediamine (3 mL) and the resulting suspension is stirred at 100° C. for 48 h. The reaction mixture is cooled to room temperature and poured onto a silica gel pad to filter. The filtered cake is rinsed with ethyl acetate (1Lx2). The combined filtrate is concentrated to dryness to give a product (6bR, 10aS)-2-oxo-2,3,6b,9,10,10a-hexahydro-1H,7H-pyrido[3′,4′:4,5] pyrrolo[1,2,3-de]quinoxaline-8-carboxylic acid ethyl esters a white solid (8g, yield 79%). MS (ESI) m/z 302.1 [M+H]⁺.

Step 3: (6bR, 10aS)-2-oxo-2,3,6b,9,10,10a-hexahydro-1H,7H-pyrido[3′,4′:4,5]pyrrolo[1,2,3-de]quinoxaline-8-carboxylic acid ethyl ester (6.4 g, 21.2 mmol) is suspended in HBr/acetic acid solution (64 mL, 33% w/w) at room temperature. The mixture is heated at 50° C. for 16 h. After cooling and treatment with ethyl acetate (300 mL), the mixture is filtered. The filter cake is washed with ethyl acetate (300 mL), and then dried under vacuum. The obtained HBr salt is then suspended in methanol (200 mL) and cooled with dry ice in isopropanol. Under vigorous stirring, ammonia solution (10 mL, 7N in methanol) is added slowly to the suspension to adjust the pH of the mixture to 10. The obtained mixture is dried under vacuum without further purification to give crude (6bR, 10aS)-2-oxo-2,3,6b,9,10,10a-hexahydro-1H,7H-pyrido[3′,4′:4,5]pyrrolo[1,2,3-de]quinoxaline (8.0 g), which is used directly in the next step. MS (ESI) m/z 230.2 [M+H]⁺.

Step 4: A mixture of (6bR,10aS)-6b,7,8,9,10,10a-hexahydro-1H-pyrido[3′,4′:4,5]pyrrolo[1,2,3-de]quinoxalin-2(3H)-one (100 mg, 0.436 mmol), 4-(3-bromopropoxy)benzonitrile (99 mg, 0.40 mmol) and KI (97 mg, 0.44 mmol) in DMF (2 mL) is bubbled with argon for 3 minutes and diisopropylethylamine (DIPEA) (80 μL, 0.44 mmol) is added. The resulting mixture is heated to 76° C. and stirred at this temperature for 2 h. The solvent is removed, and the residue is purified by silica gel column chromatography using a gradient of 0-100% mixed solvents[ethyl acetate/methanol/7N NH₃ (10:1: 0.1 v/v)] in ethyl acetate to obtain the title product as a white foam (35 mg, yield 45%). MS (ESI) m/z 389.1 [M+1]⁺. ¹H NMR (500 MHz, DMSO-d₆) δ10.3 (s, 1H), 7.8 (d, J=8.80 Hz, 2H), 7.1 (d, J=8.79 Hz, 2H), 6.8 (d, J=7.39 Hz, 1H), 6.6 (t, J=7.55 Hz, 1H), 6.6 (d, J=6.78 Hz, 1H), 4.1 (t, J=6.36 Hz, 2H), 3.8 (d, J=14.53 Hz, 1H), 3.3-3.2 (m, 3H), 3.0-2.8 (m, 1H), 2.7-2.6 (m, 1H), 2.5-2.3 (m, 2H), 2.2-2.0 (m, 1H), 2.0-1.8 (m, 3H), 1.8-1.7 (m, 1H), 1.7 (t, J=11.00 Hz, 1H).

### Example 5: Synthesis of (6bR,10aS)-8-(3-(4-chlorophenoxy)propyl)-6b,7,8,9,10,10a-hexahydro-1H-pyrido[3′,4′:4,5]pyrrolo[1,2,3-de]quinoxalin-2(3H)-one

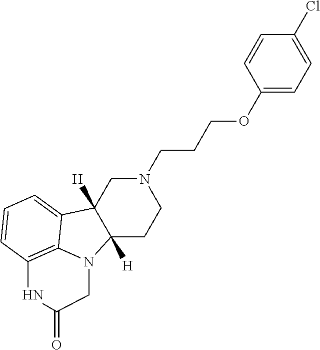 [SMILES: O=C1CN2c3c(cccc3[C@@H]3CN(CCCOc4ccc(Cl)cc4)CC[C@@H]32)N1]

To a degassed mixture of (6bR, 10aS)-6b,7,8,9,10, 10a-hexahydro-1H-pyrido[3′,4′:4,5]pyrrolo-[1,2,3-de]quinoxalin-2(3H)-one (110 mg, 0.48 mmol), 1-(3-bromopropoxy)-4-chlorobenzene (122 mg, 0.49 mmol) and KI (120 mg, 0.72 mmol) in DMF (2.5 mL) i s added DIPEA (100 μL, 0.57 mmol). The resulting mixture is heated up to 76° C. and stirred at this temperature for 2 h. The solvent is removed, and the residue is purified by silica gel column chromatography using a gradient of 0-100% mixed solvents [ethyl acetate/methanol/7N NH₃ (10:1: 0.1 v/v)] in ethyl acetate. The title product is given as a white solid (41 mg, yield 43%). (ESI) m/z 398.1 [M+1]⁺. ¹H NMR (500 MHZ, DMSO-d₆) δ10.3 (s, 1H), 7.4-7.2 (m, 2H), 6.9 (d, J=8.90 Hz, 2H), 6.8-6.7 (m, 1H), 6.6 (t, J =7.53 Hz, 1H), 6.6 (dd, J =1.04, 7.80 Hz, 1H), 4.0 (t, J=6.37 Hz, 2H), 3.8 (d, J=14.53 Hz, 1H), 3.3-3.2 (m, 3H), 2.9-2.8 (m, 1H), 2.7-2.6 (m, 1H), 2.4 (ddt, J=6.30, 12.61, 19.24 Hz, 2H), 2.1-2.0 (m, 1H), 2.0-1.9 (m, 1H), 1.9-1.7 (m, 3H), 1.7 (t, J =10.98 Hz, 1H).

### Example 6: Synthesis of (6bR,10aS)-8-(3-(quinolin-8-yloxy)propyl)-6b,7,8,9,10,10a-hexahydro-1H-pyrido[3′,4′:4,5]pyrrolo[1,2,3-de]quinoxalin-2(3H)-one

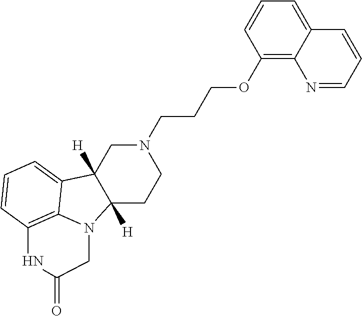 [SMILES: O=C1CN2c3c(cccc3[C@@H]3CN(CCCOc4cccc5cccnc45)CC[C@@H]32)N1]

A mixture of (6bR,10aS)-6b,7,8,9,10,10a-hexahydro-1H-pyrido[3′,4′:4,5]pyrrolo[1,2,3-de]quinoxalin-2(3H)-one (120 mg, 0.52 mmol), 8-(3-chloropropoxy)quinoline (110 mg, 0.50 mmol) and KI (120 mg, 0.72 mmol) in DMF (2.5 mL) is bubbled with argon for 3 minutes and DIPEA (100 μL, 0.57 mmol) is added. The resulting mixture is heated up to 76° C. and stirred at this temperature for 2 h. The solvent is removed, and the residue is suspended in dichloromethane (30 mL) and washed with water (10 mL). The dichloromethane phase is dried over K₂CO₃. The separated organic phase is evaporated to dryness. The residue is purified by silica gel column chromatography using a gradient of 0-100% mixed solvents [ethyl acetate/methanol/7N NH₃ (10:1: 0.1 v/v)] in ethyl acetate to produce the title product as a light brown solid (56 mg, yield 55%). (ESI) m/z 415.2 [M+1]⁺. ¹H NMR (500 MHZ, DMSO-d₆) δ10.1 (s, 1H), 8.9 (dd, J=1.68, 4.25 Hz, 1H), 8.3 (dd, J=1.71, 8.33 Hz, 1H), 7.7-7.5 (m, 3H), 7.3 (dd, J=1.50, 7.44 Hz, 1H), 7.0-6.8 (m, 1H), 6.8-6.5 (m, 2H), 4.4 (t, J=5.85 Hz, 2H), 3.9 (d, J=14.55 Hz, 1H), 3.8-3.6 (m, 2H), 3.5 (s, 1H), 3.4 (d, J=14.47 Hz, 1H), 2.9 (b, 1H), 2.3 (d, J=23.61 Hz, 5H), 1.3 (d, J=7.00 Hz, 3H).

### Example 7: Receptor Binding Profile

Receptor binding is determined for the Compound of Example 1 (the Compound of Formula A), and the Compounds of Examples 2 to 6. The following literature procedures are used, each of which reference is incorporated herein by reference in their entireties: 5-HT_{2A}: Bryant, H. U. et al. (1996), Life Sci., 15:1259-1268; D2: Hall, D. A. and Strange, P. G. (1997), Brit. J. Pharmacol., 121:731-736; D1: Zhou, Q. Y. et al. (1990), Nature, 347:76-80; SERT: Park, Y. M. et al. (1999), Anal. Biochem., 269:94-104; Mu opiate receptor: Wang, J. B. et al. (1994), FEBS Lett., 338:217-222.

In general, the results are expressed as a percent of control specific binding:

$$\frac{measured specific binding}{control specific binding} \times 100$$

and as a percent inhibition of control specific binding:

$$100 - ( \frac{measured specific binding}{control specific binding} \times 100 )$$

obtained in the presence of the test compounds.

The IC₅₀ values (concentration causing a half-maximal inhibition of control specific binding) and Hill coefficients (nH) are determined by non-linear regression analysis of the competition curves generated with mean replicate values using Hill equation curve fitting:

$$Y = D + [ \frac{A - D}{1 + {( C / {C}_{50} )}^{nH}} ]$$

where Y=specific binding, A=left asymptote of the curve, D=right asymptote of the curve, C=compound concentration, C₅₀=IC₅₀, and nH=slope factor. This analysis was performed using in—house software and validated by comparison with data generated by the commercial software SigmaPlot® 4.0 for Windows® (1997 by SPSS Inc.). The inhibition constants (Ki) were calculated using the Cheng Prusoff equation:

$$Ki = \frac{{IC}_{50}}{( 1 + L / {K}_{D} )}$$

where L=concentration of radioligand in the assay, and K_{D}=affinity of the radioligand for the receptor. A Scatchard plot is used to determine the K_{D}.

The following receptor affinity results are obtained:

| | Ki (nM) or maximum inhibition | | | | | |
|---|---|---|---|---|---|---|
| Receptor | Ex. 1 | Ex. 2 | Ex. 3 | Ex. 4 | Ex. 5 | Ex. 6 |
| 5-HT_{2A} | 8.3 | 2.6 | 3.1 | 0% @ | 15% @ | 0% @ |
| | | | | 10 nM | 10 nM | 10 nM |
| D2 | 160 | 15 | 84 | | | |
| D1 | 50 | 5.2 | 13 | 0% @ | 0% @ | 0% @ |
| | | | | 50 nM | 50 nM | 50 nM |
| SERT | 590 | 540 | | | | |
| Mu opiate receptor | 11 | 39 | 30 | 15 | 7.3 | 11 |

Additional compounds of Formula I are prepared by procedures analogous to those described in Examples 1-6. The receptor affinity results for these compounds are shown in the table below:

Compound Structure

| L | —(CH₂)ₙX— | | | | | | | | |
|---|---|---|---|---|---|---|---|---|---|
| n | 4 | 2 | 3 | 3 | 3 | 3 | 3 | 3 | 3 |
| X | O | O | O | O | O | CH₂ | NH | N(CH₃) | S |
| Z | 4-F- | 4-F- | 4-MeO- | 4-F- | 4-F- | 4-F- | 4-F- | 4-F- | 4-F- |
| | phenyl | phenyl | phenyl | 3-OH- | 2-OH- | phenyl | phenyl | phenyl | phenyl |
| | | | | phenyl | phenyl | | | | |
| R¹ | H | H | H | H | H | H | H | H | H |
| R², R³ | H, H | H, H | H, H | H, H | H, H | H, H | H, H | H, H | H, H |
| Receptor | Ki (nM) or maximum inhibition | | | | | | | | |
| 5-HT_{2A} | 37% @ | 48% @ | 0% @ | 110 | 19 | 85% @ | 32% @ | 76% @ | 93% @ |
| | 100 nM | 100 nM | 100 nM | | | 100 nM | 100 nM | 100 nM | 100 nM |
| D2 | 27% @ | 24% @ | | 0% @ | 67 | 24% @ | 25% @ | 14% @ | 49% @ |
| | 100 nM | 100 nM | | 100 nM | | 100 nM | 100 nM | 100 nM | 100 nM |
| D1 | 5.4% @ | 10% @ | 0% @ | 25% @ | 22% @ | 32% @ | 11% @ | 21% @ | 54% @ |
| | 100 nM | 50 nM | 100 nM | 100 nM | 100 nM | 100 nM | 100 nM | 100 nM | 100 nM |
| SERT | 3.3% @ | 0% @ | 10% @ | 13% @ | 5% @ | 16% @ | 0% @ | 53% @ | 0% @ |
| | 100 nM | 100 nM | 100 nM | 100 nM | 200 nM | 200 nM | 100 nM | 200 nM | 200 nM |
| Mu | 39% @ | 30% @ | 0% @ | 23% @ | 22% @ | 89% @ | 60% @ | 22% @ | 60% @ |
| | 100 nM | 30 nM | 100 nM | 100 nM | 100 nM | 100 nM | 100 nM | 100 nM | 100 nM |

### Example 8: DOI-induced Head Twitch Model in Mice

R-(−)-2,5-dimethoxy-4-iodoamphetamine (DOI) is an agonist of the serotonin 5-HT₂ receptor family. When administered to mice, it produces a behavioral profile associated with frequent head twitches. The frequency of these head twitches during a predetermined period of time can be taken as an estimate of 5-HT₂ receptor agonism in the brain. Conversely, this behavioral assay can be used to determine 5-HT₂ receptor antagonism in the brain by administering the DOI with or without an antagonist and recording the reduction in DOI-induced head twitches after the administration of the antagonist.

The method of Darmani et al., Pharmacol Biochem Behav. (1990) 36:901-906 (the contents of which are incorporated by reference in their entirety) is used with some modifications. (±)-DOI HCl is injected subcutaneously and the mice are immediately placed in a conventional plastic cage. The number of head twitches is counted during 6 min, beginning 1 min after DOI administration. The tested compound is administered orally 0.5 hr before the injection of DOI. Results area calculated as the EC₅₀ for reducing DOI-induced head twitches. The results are shown in the following Table:

| Compound | EC₅₀ (mg/kg, p.o.) |
|---|---|
| Example 1 | 0.44 |

The results show that the compound of Example 1 potently blocks DOI head twitch, consistent with the in-vitro 5-HT_{2A} results shown in Example 7.

### Example 9: Mouse Tail Flick Assay

The Mouse Tail Flick Assay is a measure of analgesia, indicated by the pain reflex threshold of restrained mice. Male CD-1 mice are positioned with their tails under a focused beam of a high-intensity infrared heat source, resulting in heating of the tail. The animal can withdraw its tail from the heat source at any time that it becomes uncomfortable. The amount of time (latency) between turning on the heating instrument and the flicking of the mouse's tail out of path of the heat source is recorded. Administration of morphine results in analgesia, and this produces a delay in the mouse's reaction to the heat (increased latency). Prior administration of a morphine receptor (MOR) antagonist, i.e., naloxone (NAL), reverses the effect and results in normal latency time. This test is used as a functional assay to gauge antagonism of mu-opiate receptors.

### Example 9a: Antagonism of Morphine-Induced Analgesia by Compound of Example 1

Ten male CD-1 mice (about 8 weeks of age) are assigned to each of five treatment groups. The groups are treated as follows: Group (1) [negative control]: administered 0.25% methylcellulose vehicle p.o., 60 minutes before the tail flick test, and saline vehicle 30 minutes before the tail flick test; Group (2) [positive control]: administered 0.25% methylcellulose vehicle p.o., 60 minutes before the test, and 5 mg/kg morphine in saline 30 minutes before the test; Group (3) [positive control]: administered 3 mg/kg naloxone in saline 50 minutes before the test, and 5 mg/kg morphine in saline 30 minutes before the test; Groups (4)-(6): administered either 0.1 mg/kg, 0.3 mg/kg or 1 mg/kg of the test compound in 0.25% methylcellulose vehicle p.o., 60 minutes before the test, and 5 mg/kg morphine in 30 minutes before the test. The results are shown in the following table as mean latency measured in seconds:

| | | | | Group 4 | Group 5 | Group 6 |
|---|---|---|---|---|---|---|
| | | | | Cmpd/ | Cmpd/ | Cmpd/ |
| | Group | Group | Group | Mor | Mor | Mor |
| | 1 | 2 | 3 | (0.1 | (0.3 | (1 |
| | Veh/Veh | Veh/Mor | Nal/Mor | mg/kg) | mg/kg) | mg/kg) |
| Ex. 1 | 0.887 | 8.261 | 3.013 | 6.947 | 5.853 | 6.537 |

The results demonstrate that the compound of Example 1 exerts a dose-dependent blockade of morphine-induced mu-opiate receptor activity.

### Example 9b: Analgesia by Compound of Example 1, Inhibited by Naloxone

In a second study using the mouse tail flick assay as described above, the compound of Example 1 is further compared at doses of 1.0 mg/kg, 3.0 mg/kg and 10 mg/kg against morphine at 5 mg/kg with and without pre-dosing with naloxone at 3 mg/kg (intraperitoneal). In the pre-treatment groups, the naloxone is administered 20 minutes prior to the tail flick test. In the non-pre-treatment controls, saline is administered 20 minutes prior to the tail flick test. In each group, the vehicle, morphine or compound of Example 1 is administered 30 minutes before the tail flick test. The results are shown in the table below as mean latency in seconds:

| | | | Ex. 1 | Ex. 1 | Ex. 1 |
|---|---|---|---|---|---|
| | | | at | at | at 10 |
| | Vehicle | Morphine | 1 mg/kg | 3 mg/kg | mg/kg |
| Saline pre- | 0.9 | 9.8 | 4.1 | 7.4 | 9.8 |
| treatment | | | | | |
| Naloxone pre- | 0.8 | 1.5 | 1.3 | 1.7 | 2.1 |
| treatment | | | | | |

It is found that administration of the compound of Example 1 at all doses significantly increased the latency to tail flick, and that this effect is attenuated by pre-treatment with naloxone. This result demonstrates a dose-dependent analgesic effect produced by the Compound of Example 1, and further suggests that this effect is mediated by mu-opioid receptor agonism.

### Example 9c: Time Course for Analgesia, Compound of Example 1

The tail flick assay as described above is repeated to determine the time course of analgesia resulting from administration of the compound of Example 1. Mice are administered s.c. either (1) vehicle 30 minutes prior to assay, (2) 5 mg/kg morphine 30 minutes prior to assay, or (3)-(7) the 1 mg/kg of compound of Example 3 30 minutes, 2 hours, 4 hours, 8 hours or 24 hours prior to assay. The results are shown in the table below as mean latency in seconds:

| Treatment | TF Latency (s) |
|---|---|
| Vehicle, 30 min prior | 1.30 |
| Morphine, 30 min prior | 7.90 |
| Cmpd. Ex. 1, 30 min prior | 5.77 |
| Cmpd. Ex. 1, 2 h prior | 2.42 |
| Cmpd. Ex. 1, 4 h prior | 1.48 |
| Cmpd. Ex. 1, 6 h prior | 1.36 |
| Cmpd. Ex. 1, 24 h prior | 1.29 |

The results show that the Compound of Example 1 produces effective analgesia when administered 30 minutes or 2 hours prior to the tail flick assay (ANOVA, P<0.001 vs. vehicle). When administered 4 hours, 8 hours, or 24 hours prior to the tail flick assay, the compound of Example 1 at 1 mg/kg does not produce an analgesic effect significantly different from the vehicle control. Thus, the compound of Example 1 does not produce prolonged analgesia, which means that it would have a lower potential for abuse and a lower risk of drug-drug interactions compared to other opiate analgesics.

### Example 9d: Analgesia from Chronic Administration of the Compound of Example 1

The tail flick assay described above is repeated using a test model in which animals receive a 14-day chronic treatment regimen, followed by an acute treatment 30 minutes prior to the tail flick assay. The mice are divided into three broad groups with six sub-groups of 10 mice each. The three groups receive as the chronic treatment either (A) vehicle, (B) compound of Example 1 at 0.3 mg/kg, or (C) compound of Example 2 at 3.0 mg/kg. Each sub-group further receives as the acute treatment either (1) vehicle, or (2)-(6) the compound of Example 1 at 0.01, 0.03, 0.1, 0.3 or 1.0 mg/kg. All treatments are administered s.c. The results are shown in the table below as mean latency to tail flick in seconds:

| Group | Chronic Treatment | Acute Treatment | Latency (s) |
|---|---|---|---|
| (A) | Vehicle | Vehicle | 1.09 |
| | Vehicle | Ex. 1, 0.01 mg/kg | 1.87 |
| | Vehicle | Ex. 1, 0.03 mg/kg | 2.50 |
| | Vehicle | Ex. 1, 0.1 mg/kg | 5.26 |
| | Vehicle | Ex. 1, 0.3 mg/kg | 8.26 |
| | Vehicle | Ex. 1, 1.0 mg/kg | 9.74 |
| (B) | Ex. 3, 0.3 mg/kg | Vehicle | 0.893 |
| | Ex. 3, 0.3 mg/kg | Ex. 1, 0.01 mg/kg | 1.66 |
| | Ex. 3, 0.3 mg/kg | Ex. 1, 0.03 mg/kg | 1.30 |
| | Ex. 3, 0.3 mg/kg | Ex. 1, 0.1 mg/kg | 2.60 |
| | Ex. 3, 0.3 mg/kg | Ex. 1, 0.3 mg/kg | 3.93 |
| | Ex. 3, 0.3 mg/kg | Ex. 1, 1.0 mg/kg | 5.64 |
| (C) | Ex. 3, 3.0 mg/kg | Vehicle | 1.04 |
| | Ex. 3, 3.0 mg/kg | Ex. 1, 0.01 mg/kg | 1.64 |
| | Ex. 3, 3.0 mg/kg | Ex. 1, 0.03 mg/kg | 1.80 |
| | Ex. 3, 3.0 mg/kg | Ex. 1, 0.1 mg/kg | 3.94 |
| | Ex. 3, 3.0 mg/kg | Ex. 1, 0.3 mg/kg | 4.84 |
| | Ex. 3, 3.0 mg/kg | Ex. 1, 1.0 mg/kg | 7.94 |

It is found that 0.1, 0.3 and 1.0 mg/kg acute treatment with the compound of Example 1 produces a statistically significant dose-dependent analgesic effect compared to in-group acute treatment with vehicle. This is true for each of the chronic groups (A), (B) and (C). As compared to pre-treatment with vehicle, pre-treatment with the compound of Example 1 at 0.3 mg/kg or 3.0 mg/kg generally showed a statistically significant decrease in tail flick latency when the same acute treatment subgroups are compared. These results demonstrate that while some tolerance to the analgesic effect of the compound of Example 1 occurs after 14-days of chronic treatment, the analgesia obtained remains effective despite chronic pre-treatment.

### Example 10: CNS Phosphoprotein Profile

A comprehensive molecular phosphorylation study is also carried out to examine the central nervous system (CNS) profile of the compound of Example 1. The extent of protein phosphorylation for selected key central nervous system proteins is measured in mice nucleus accumbens. Examined proteins include ERK1, ERK2, Glu1, NR²B and TH (tyrosine hydroxylase), and the compound of Example 1 is compared to the antipsychotic agents risperidone and haloperidol.

Mice were treated with the compound of Example 1 at 3 mg/kg, or with haloperidol at 2 mg/kg. Mice were killed 30 minutes to 2 hours post-injection by focused microwave cranial irradiation, which preserves brain phosphoprotein as it exists at the time of death. Nucleus accumbens was then dissected from each mouse brain, sliced and frozen in liquid nitrogen. Samples were further prepared for phosphoprotein analysis via SDS-PAGE electrophoresis followed by phosphoprotein-specific immunoblotting, as described in Zhu H, et al., Brain Res. 2010 Jun. 25; 1342:11-23. Phosphorylation at each site was quantified, normalized to total levels of the protein (non-phosphorylated), and expressed as percent of the level of phosphorylation in vehicle-treated control mice.

The results demonstrate that the compound of Example 1 has no significant effect on tyrosine hydroxylase phosphorylation at Ser40 at 30 minutes or 60 minutes, in contrast to haloperidol which produces a greater than 400% increase, and risperidone which produces a greater than 500% increase, in TH phosphorylation. This demonstrates that the Compounds of the invention do not disrupt dopamine metabolism.

The results further demonstrate that the compound of Example 1 has no significant effect on NR²B phosphorylation at Tyr1472 at 30-60 minutes. The compounds produce a slight increase in GluR¹ phosphorylation at Ser845, and a slight decrease in ERK2 phosphorylation at Thr183 and Tyr185. Protein phosphorylation at various sites in particular proteins are known to be linked to various activities of the cell such as protein trafficking, ion channel activity, strength of synaptic signaling and changes in gene expression. Phosphorylation the Tyr1472 in the NMDA glutamate receptor has been shown to be essential for the maintenance of neuropathic pain. Phosphorylation of Ser845 of the GluR¹ AMPA type glutamate receptor is associated with several aspects of strengthening synaptic transmission and enhanced synaptic localization of the receptor to support long term potentiation associated with cognitive abilities. It has also been reported that phosphorylation of this residue results in an increased probability of channel opening. Phosphorylation of ERK2 kinase, a member of the MAP kinase cascade, at residues T183 and Y185 is required for full activation of this kinase, ERK2 is involved in numerous aspects of cell physiology including cell growth, survival and regulation of transcription. This kinase has been reported to be important in synaptogenesis and cognitive function.

### Example 11: Mu-Opiate Receptor Activity Assays

The compound of Example 1 is tested in CHO-K1 cells expressing hOP3 (human mu-opiate receptor μ1 subtype) using an HTRF-based cAMP assay kit (cAMP Dynamic2 Assay Kit, from Cisbio, #62AM4PEB). Frozen cells are thawed in a 37°° C. water bath and are resuspended in 10 mL of Ham's F-12 medium containing 10% FBS. Cells are recovered by centrifugation and resuspended in assay buffer (5 nM KCl, 1.25 mM MgSO₄, 124 mM NaCl, 25 mM HEPES, 13.3 mM glucose, 1.25 mM KH₂PO₄, 1.45 mM CaCl₂, 0.5 g/L protease-free BSA, supplemented with 1 mM IBMX). Buprenorphine, a mu-opiate receptor partial agonist, and naloxone, a mu-opiate receptor antagonist, and DAMGO, a synthetic opioid peptide full agonist, are run as controls.

For agonist assays, 12 μL of cell suspension (2500 cells/well) are mixed with 6μL forksolin (10 μM final assay concentration), and 6μL of the test compound at increasing concentrations are combined in the wells of a 384-well white plate and the plate is incubated for 30 minutes at room temperature. After addition of lysis buffer and one hour of further incubation, CAMP concentrations are measured according to the kit instructions. All assay points are determined in triplicate. Curve fitting is performed using XLfit software (IDBS) and EC₅₀ values are determined using a 4-parameter logistic fit. The agonist assay measures the ability of the test compound to inhibit forskolin-stimulated cAMP accumulation.

For antagonist assays, 12 uL of cell suspension (2500 cells/well) are mixed with 6μL of the test compound at increasing concentrations, and combined in the wells of a 384-well white plate and the plate is incubated for 10 minutes at room temperature. 6μL of a mixture of DAMGO (D-Ala²-N-MePhe⁴-Gly-ol-enkephelin, 10 nM final assay concentration) and forksolin (10 μM final assay concentration) are added, and the plates are incubated for 30 minutes at room temperature. After addition of lysis buffer, and one hour of further incubation, cAMP concentrations are measured according the kit instructions. All assay points are determined in triplicate. Curve fitting is performed using XLfit software (IDBS) and IC₅₀ values are determined using a 4-parameter logistic fit. Apparent dissociation constants (KB) are calculated using the modified Cheng-Prusoff equation. The antagonist assay measures the ability of the test compound to reverse the inhibition of forskolin-induced cAMP accumulation caused by DAMGO.

The results are shown in the Table below. The results demonstrate that the compound of Example 1 is a weak antagonist of the Mu receptor, showing much higher IC₅₀ compared to naloxone, and that it is a moderately high affinity, but partial agonist, showing only about 22% agonist activity relative to DAMGO (as compared to about 79% activity for buprenorphine relative to DAMGO). The compound of Example 1 is also shown to have moderately strong partial agonist activity.

| | Antagonist | Agonist | |
|---|---|---|---|
| Compound | IC₅₀ (nM) | EC₅₀ (nM) | K_{B} (nM) |
| Naloxone | 5.80 | — | 0.65 |
| DAMGO | — | 1.56 | — |
| Buprenorphine | — | 0.95 | — |
| Cmpd. Ex. 1 | 641 | 64.5 | 71.4 |

Buprenorphine is a drug used for chronic pain treatment and for opiate withdrawal, but it suffers from the problem that users can become addicted due to its high partial agonist activity. To offset this, the commercial combination of buprenorphine with naloxone is used (sold as Suboxone). Without being bound by theory, it is believed that the compounds of the present invention, which are weaker partial Mu agonists than buprenorphine, with some moderate antagonistic activity, will allow a patient to be more effectively treated for pain and/or opiate withdrawal with lower risks of addiction.

In additional related study using a recombinant human MOP-beta-arresting signaling pathway, it is found that the Compound of Example 1 does not stimulate beta-arrestin signaling via the MOP receptor at concentrations up to 10 μM, but that it is an antagonist with an IC₅₀ of 0.189 μM. In contrast, the full opioid agonist Met-enkephalin stimulates beta-arrestin signaling with an EC₅₀ of 0.08 μM.

### Example 12: Rat Tolerance/Dependence Study

The compound of Example 1 is assessed during repeated (28 day) daily subcutaneous administration to male Sprague-Dawley rats to monitor drug effects on dosing and to determine if pharmacological tolerance occurs. In addition, behavioral, physical and physiological signs in the rats is monitored following abrupt cessation of repeated dosing to determine whether the compound induces physical dependence on withdrawal. Further, a pharmacokinetic study is performed in parallel with the tolerance and dependence study to determine the plasma drug exposure levels of the compound at the specific doses used in the tolerance and dependence study. Morphine is used as a positive control to ensure validity of the model and as a reference comparator from a similar pharmacological class.

The compound of Example 1 is evaluated at two doses, 0.3 and 3 mg/kg, administered subcutaneously four times per day. Repeated administration is found to produce peak plasma concentrations of 15 to 38 ng/ml (average, n=3) for 0.3 mg/kg dosing, and 70 to 90 ng/ml (average, n=3) for 3 mg/kg dosing. Peak concentration is reached at 30 minutes to 1.5 hours post-administration with comparable results obtained on the 1st, 14th and 28th day of administration.

At both doses of the compound of Example 1, it is found that there is no significant effect on animal body weight, food and water intake or body temperature during either the on-dose or withdrawal phase. The predominant behavioral and physical effects caused by repeated administration at 0.3 mg/kg is found to be hunched posture, Straub tail and piloerection during the dosing phase. At the higher dose, the main behavioral and physical signs observed are hunched posture, subdued behavior, Straub tail, tail rattle and piloerection.

A similar profile of behavioral and physical signs is observed following abrupt cessation of the compound on Day 28 of the study. While rearing and increased body tone were not observed during the on-dose phase for at 0.3 mg/kg, it is found to be significantly increased during the withdrawal phase. At the higher dose, mild rearing is observed during the on-dose phase, but during the withdrawal phase, rearing is more pronounced and increased body tone is observed.

As a positive control, morphine is doses at 30 mg/kg orally twice per day. This dosing regimen, as expected, is observed to be associated with changes in body weight, food and water intake, rectal temperature and clinical signs consistent with the development of tolerance and withdrawal-induced dependence. Body weight was significantly increased compared with the vehicle-treated control group on Days 2 and 3, while it was significantly decreased from Day 5. Morphine decreased food intake significantly on Days 1-9. Thereafter food intake is generally observed to be lower than for the control group, but was not significantly different from controls on Days 9, 13, 14 16, 18, 21, 22 and Day 25. These effects on body weight and food intake demonstrate tolerance to the effect of morphine.

Water intake of the morphine-treated group is also found to be significantly lower than the control group on 25 out of 28 days during the on-dose phase. Body temperature is also generally lower than the control group during the on-dose phase, significantly so on Days 20, 21 and 27. The predominant behavioral effects induced by morphine during the on-dose phase are observed to be Straub tail, jumping, digging, increased body tone, increased locomotor activity, explosive movements and exopthalmus.

Furthermore, withdrawal of morphine administration on Day 28 is observed to result in an initial further decrease in food intake followed by rebound hyperphagia, with significantly increased food intake on Day 33 versus the control group. Food intake returns to control levels by Day 35. Similarly, rats which had previously received morphine also are observed to have an initial reduction in water intake on Day 29, followed by rebound hyperdipsia (water consumption returns to control levels by Day 31). In addition, statistically significant decreases in rectal body temperature are observed during dosing, but body temperature returns to control levels during the withdrawal phase.

Moreover, new behavioral and physical signs are observed during the withdrawal phase from morphine, and this demonstrates the presence of dependence. These signs include piloerection, ataxia/rolling gait, wet dog shakes and pinched abdomen. Other abnormal behaviors observed during the on-dose phase gradually disappear during the withdrawal phase. By Day 35, rearing was the only behavior or physical sign observed with high incidence in the rats that had previously received morphine.

Thus, repeated morphine administration is shown to produce clear signs of tolerance and dependence in this study, with changes in body weight, food and water intake, rectal temperature and clinical signs consistent with the development of tolerance and withdrawal induced dependence. This demonstrates the validity of the study method in detecting physiological alterations during administration and cessation of dosing.

In contrast, repeated administration of the Compound of Example 1, at both 0.3 and 3 mg/kg four times, does not produce tolerance during subcutaneous dosing for 28 days. Furthermore, on withdrawal, a similar but decreasing profile of behavioral and physical signs is observed at the highest dose, which is not considered to be of clinical significance. Thus, overall, the Compound of Example 1 was found not to produce a syndrome of physical dependence upon cessation of dosing.

### Example 13: Oxycodone-Dependent Withdrawal Study in Mice

Oxycodone is administered to male C₅₇BL/6J mice for 8 days at an increasing dose regimen of 9, 17.8, 23.7, and 33 mg/kg b.i.d. (7 hours between injections) on days 1-2, 3-4, 5-6 and 7-8 respectively. On the morning of the ninth day, the mice are administered the compound of Example 1 at either 0.3, 1 or 3 mg/kg subcutaneous. This is followed 30 minute later by either an injection of vehicle or with an injection of 3 mg/kg of naloxone. Another cohort of mice serve as negative controls, and instead of oxycodone, these mice are administered saline on days 1 to 8. On day 9, these mice are administered either vehicle (followed by naloxone, as above) or the compound of Example 1 at 3 mg/kg, s.c. (followed by naloxone, as above).

On day 9, immediately after the injection of naloxone (or vehicle), the mice are individually placed in clear, plastic cages and are observed continuously for thirty minutes. The mice are monitored for common somatic signs of opiate withdrawal, including jumping, wet dog shakes, paw tremors, backing, ptosis, and diarrhea. All such behaviors are recorded as new incidences when separated by at least one second or when interrupted by normal behavior. Animal body weights are also recorded immediately before and 30 minutes after the naloxone (or vehicle) injections. Data is analyzed with ANOVA followed by the Tukey test for multiple comparisons, when appropriate. Significant level is established at p<0.05.

The results are shown in the Table below:

| Dosing: (1) on days 1-8, | Total | | | Body |
|---|---|---|---|---|
| (2) on day 9, followed by | Number | Paw | | Weight |
| (3) 30 minutes later | of Signs | Tremors | Jumps | Loss |
| (1) Saline; (2) Vehicle, | 2.2 | 0.87 | 0 | 0.5% |
| (3) Naloxone | | | | |
| (1) Saline; (2) Compound | 5.3 | 0.12 | 0 | 0.4% |
| 3.0 mg/kg, (3) Naloxone | | | | |
| (1) Oxycodone; (2) | 155.1 | 73.6 | 63.2 | 7.8% |
| Compound 3.0 mg/kg, | | | | |
| (3) Vehicle | | | | |
| (1) Oxycodone; (2) | 77.5 | 19.6 | 40.6 | 7.5% |
| Compound 0.3 mg/kg, | | | | |
| (3) Naloxone 3 mg/kg | | | | |
| (1) Oxycodone; (2) | 62.5 | 14.8 | 34.8 | 6.0% |
| Compound 1.0 mg/kg, | | | | |
| (3) Naloxone 3 mg/kg | | | | |
| (1) Oxycodone; (2) | 39.5 | 0.5 | 26.6 | 4.0% |
| Compound 3.0 mg/kg, | | | | |
| (3) Naloxone 3 mg/kg | | | | |

Total number of signs includes paw tremors, jumps, and wet dog shakes. In oxycodone-treated mice, it is found that naloxone elicits a significant number of total signs, paw tremors, jumps and body weight change (p≤0.0001 for each). At all doses tested, the compound of Example 1 produces a significant decrease in total number of signs and paw tremors. In addition, at 3.0 mg/kg, the compound also produces a significant decrease in jumps and attenuated body weight loss.

These results demonstrate that the compound of Example 1 dose-dependently reduces the signs and symptoms of opiate withdrawal after the sudden cessation of opiate administration in opiate-dependent rats.

### Example 14: Formalin Paw Test (Inflammatory Pain Model)

Sub-plantar administration of chemical irritants, such as formalin, causes immediate pain and discomfort in mice, followed by inflammation. Subcutaneous injection of 2.5% formalin solution (37 wt % aqueous formaldehyde, diluted with saline) into the hind paw results in a biphasic response: an acute pain response and a delayed inflammatory response. This animal model thus provides information on both acute pain and sub-acute/tonic pain in the same animal.

C₅₇ mice are first habituated in an observation chamber. 30 minutes prior to formalin challenge, mice are administered either vehicle injected subcutaneously, 5 mg/kg of morphine (in saline) injected subcutaneously, or the compound of Example 1 (in 45% w/v aqueous cyclodextrin) injected subcutaneously at either 0.3, 1.0 or 3.0 mg/kg. In addition, another set of mice are treated with the control vehicle or the compound of Example 1 at 3.0 mg/kg, via oral administration, rather than subcutaneous injection.

The mice are then given a subcutaneous injection into the plantar surface of the left hind paw of 20 μL of 2.5% formalin solution. Over the next 40 minutes, the total time spent licking or biting the treated hind-paw is recorded. The first 10 minutes represent the acute nociceptic response, while the latter 30 minutes represents the delayed inflammatory response. At one minter intervals, each animal's behavior is assessed using “Mean Behavioral Rating,” which is scored on a scale of 0 to 4:
0: no response, animal sleeping
1: animal walking lightly on treated paw, e.g., on tip-toe
2: animal lifting treated paw
3: animal shaking treated paw
4: animal licking or biting treated paw
   Data are analyzed by ANOVA followed by post-hoc comparisons with Fisher tests, where appropriate. Significance is established at p<0.05.

The results are shown in the Table below.

| | Mean Behavior | | | | Mean Licking | | | |
|---|---|---|---|---|---|---|---|---|
| | Rating (0-4) | | | | Time (min) | | | |
| | 0-10 | 11-40 | 0-6 | 16-40 | 0-10 | 11-40 | 0-6 | 16-40 |
| | Min | min | min | min | min | min | min | min |
| Vehicle | 1.4 | 1.4 | 2.1 | 1.5 | 34 | 75 | 32 | 76 |
| (SC) | | | | | | | | |
| Vehicle | 1.2 | 0.9 | 1.9 | 1.0 | 29 | 50 | 33 | 40 |
| (PO) | | | | | | | | |
| Morphine | 1.1 | 0.2 | 1.7 | 0.2 | 11 | 0 | 11 | 0 |
| Cmpd, SC | 1.5 | 1.0 | 2.3 | 1.2 | 31 | 68 | 34 | 70 |
| 0.3 mg/kg | | | | | | | | |
| Cmpd, SC | 1.3 | 1.0 | 1.9 | 1.1 | 26 | 60 | 26 | 65 |
| 1.0 mg/kg | | | | | | | | |
| Cmpd, SC | 0.8 | 0.1 | 1.3 | 0.1 | 14 | 36 | 11 | 36 |
| 3.0 mg/kg | | | | | | | | |
| Cmpd, PO | 0.9 | 0.8 | 1.5 | 0.9 | 11 | 3 | 9 | 3 |
| 3.0 mg/kg | | | | | | | | |

The results demonstrate a significant treatment effect during both the early phase (0-10 min) and late phase (11-40 min) response periods. Post-hoc comparisons show that, compared to vehicle treatment, subcutaneous injection of morphine or the compound of Example 1 (at 3 mg/kg) significantly attenuates the pain behavior rating induced by formalin injection, as well as significantly reducing licking time. Post-hoc comparisons also show that subcutaneous injection of morphine or the compound of Example 1 (at 3 mg/kg), as well as the compound of Example 1 orally (at 3 mg/kg), significantly reduces time spent licking. While the mean pain behavior rating was also reduced using 1.0 mg/kg of compound subcutaneous and at 3.0 mg/kg oral, these effects were not statistically significant in this study. Licking time was similarly reduced using 1.0 mg/kg of the compound of Example 1 subcutaneously, but the result was not statistically significant in this study. It was also found that no mice in the study underwent significant changes in body weight in any of the study groups.

### Example 15: Self Administration in Heroin-Maintained Rats

A study is performed to determine whether heroin-addicted rats self-administer the compound of Example 1, and it is found that they do not, further underscoring the non-addictive nature of the compounds of the present disclosure.

The study is performed in three stages. In the first stage, rats are first trained to press a lever for food, and they are then provided with an in-dwelling intravenous jugular catheter and trained to self-administer heroin. In response to a cue (the lighting of a light in the cage), three presses of the lever by the animal results in a single heroin injection via the catheter. The heroin is provided at an initial dose of 0.05 mg/kg/injection, and later increased to 0.015 mg/kg/injection. This trained response is then extinguished by replacing the heroin supply with saline. In the second phase, the saline solution is replaced by a solution of the compound of Example 1, at one of four doses: 0.0003 mg/kg/injection, 0.001 mg/kg/injection, 0.003 mg/kg/injection, and 0.010 mg/kg/injection. Each individual rat is provided with either one or two different doses of the compound in rising fashion. This response is then extinguished with saline injections, followed by the third phase, which repeats the use of heroin at 0.015 mg/kg/injection. The purpose of the third phase is to demonstrate that the rats still show addictive behavior to heroin at the end of the study. The study results are shown in the table below:

| | | Mean Lever |
|---|---|---|
| Treatment | Animals (n) | presses |
| Saline Extinction 1 | 21 | 4.08 |
| Heroin Acquisition | 21 | 19.38* |
| (0.015 mg/kg/inj) | | |
| Cmpd. Ex. 1 at 0.0003 mg/kg/inj | 8 | 3.17** |
| Cmpd. Ex. 1 at 0.0003 mg/kg/inj | 8 | 3.29** |
| Cmpd. Ex. 1 at 0.0003 mg/kg/inj | 8 | 3.99** |
| Cmpd. Ex. 1 at 0.0003 mg/kg/inj | 8 | 4.87** |
| Saline Extinction 2 | 19 | 3.60** |
| Heroin Reinstatement | 19 | 17.08** |
| (0.015 mg/kg/inj) | | |

*P < 0.001 for heroin acquisition vs. saline extinction 1 (multiple t test);
**P < 0.001 for Cmpd of Ex. 1 vs. heroin acquisition (Dunnett's test); P >0.7 for all comparisons between Cmpd. of Ex. 1 and saline extinction 1 (William's test)

The results demonstrate that there is a statistically significant increase in lever pressing by the rats when being administered heroin, but that there was no significant difference when being administered saline or the compound of Example 1. Thus, the results suggest that rats do not become addicted to the compound of Example 1.

It should be noted that this study uses the term “reinstatement” to show that the rats, which had not shown interest in self-administering the compound of Example 1, do self-administer heroin if it is made available. As such, “reinstatement” here means that the animals have retained their ability or training to intravenously self-administer heroin. However, the study results show that rats under these circumstances do not choose to self-administer the compound of Example 1, demonstrating that it is not psychologically rewarding to the rats (i.e., not psychologically addictive).

### Example 16: Rat Models of Neuropathic Pain

The compound of Example 1 is also tested in an STZ-rat model of neuropathic pain. Briefly, adult female rats are made diabetic by treatment with streptozotocin (STZ), an alkylating neoplastic agent which is particularly toxic to the insulin-producing beta cells of the pancreas. The resulting type-I diabetes in the rats leads to the development of diabetic neuropathy over a 3 to 6-week period. This can be demonstrated using various indices of painful neuropathy, such as allodynia to light touch, hyperalgesia to pressure, cold heat and chemical stimuli. Once diabetic neuropathy has been induced, the rats may be treated to determine the analgesic effect of compounds.

Paw tactile response threshold is a clinical assessment of allodynia to light touch. It can be measured using manual von Frey filaments (as described in Otto et al., Pain, 101:187-92 (2003). A series of von Frey filaments with logarithmically increasing stiffness are used, and the rats' response to each filament is observed. The results are used to calculate a 50%-withdrawal threshold (an amount of stiffness in the filament resulting in a 50% probability of withdrawal of the rat's paw).

Paw mechanical response threshold is also a clinical assessment of allodynia to light touch, but it relies on the observed response to pressure (force) applied to a paw.

Paw cold response threshold is a clinical assessment of cold pain perception. A rigid filament with a thermoelectric cooling system is used to stimulate the plantar surface of the hind paw for 5 seconds. The stimulus is repeated ten times at 2 to 5-minute intervals. The number of paw withdrawal responses is recorded and converted to a response frequent figure (%). This procedure is repeated at a variety of temperatures. In diabetic rats, it is found that below a certain threshold temperature, an enhanced (hyperalgesic) response to cold is observed in the rats. At a stimulus temperature of 20° C. or 15° C., the response frequency is found to be substantially the same between STZ-treated rats and control rats (from about 10-20% response). In contrast, at a stimulus temperature of 10° C. or below, there is a substantial divergence of frequency response. In control rats, a stimulus temperature of 10° C. results in about 10% response frequency, while at 5° C., this increases to about 40% response frequency. In contrast, STZ-treated rats show a response frequency of about 60% at 10° C., and about 80% at 5° C. This demonstrates that the STZ treated rats suffer from cold hyperalgesia at a temperature of 10° C. or below. A stable cold allodynia response is observed 4-12 weeks after induction of diabetes.

### Example 16a: The Compound of Example 1 Suppresses Cold Allodynia Response

Six groups of rats are compared over a six-hour period from injection (sub-cutaneous) of the Compound of Example 1 (“Compound”) or vehicle: (1) Control rats injected with vehicle, (2) Control rats injected with 10 mg/kg of Compound, (3) STZ-diabetic rats injected with vehicle, (4) STZ-diabetic rats injected with 1 mg/kg of Compound, (5) STZ-diabetic rats injected with 3 mg/kg of Compound, and (6) STZ-diabetic rats injected with 10 mg/kg of Compound. The cold allodynia response test is performed at 0 hours, 1 hour, 2 hours, 4 hours and 6 hours, as described above, using a 10° C. stimulus temperature. The injection vehicle is pure polyethylene glycol-400 (PEG400).

The results show that control group rats (1) and (2) display a response frequency between 10 and 30% at all time points (normal cold response). The positive vehicle control rats of Group (3) display a response frequency of 70-90% at all time, demonstrating cold allodynia. Comparison of Groups (4) to (6) shows a dose-dependent reduction in cold allodynia. At 1 mg/kg (Group (4)), the Compound reduces the response frequency to near normal at 1 hour (˜35% response), and this decays back to about 70% at 4 hours and about 75% at 6 hours. At 3 mg/kg (Group (5)), the Compound reduces the response frequency to normal levels at 1 hour (about 10% response), and this decays back to about 65% at 4 hours and 75% at 6 hours. At 10 mg/kg (Group (6)), the Compound reduces the response frequency to the normal range at 1 hour (about 15%), and it remains in the normal range at 2 hours and 4 hours (about 10% at 2 hours, about 20% at 4 hours), rising to only about 40% at 6 hours.

Rats are also tested in the rotorod motor coordination model at a dose of 3 mg/kg, and no motor incoordination is found to result from the Compound. This further supports that the cold allodynia observations are due to pain inhibition rather than due to delayed motor response to the cold stimulus. Interestingly, the time frame of duration of action of the subcutaneous injection of the Compound is consistent with the results obtained in the mouse tail flick assay (Example 9c).

### Example 16b: The Compound of Example 1 Suppresses Hyperalgesia in Response to Tactile Stimuli

Similar to the observations noted in Example 16a, STZ-treated diabetic rats demonstrate stable tactile hyperalgesia, as measured after manual application of von Frey filaments, when tested 4-12 weeks after induction of diabetes.

Rats are divided into six groups as described in Example 16a and are monitored for 6-hours after administration of the Compound of Example 1 or vehicle. This test is performed using the von Frey filaments as described above.

The results show that the control animals of both Group (1) and Group (2) display a 50% withdrawal threshold of 8 to 14 grams, which is within the normal range. In contrast, the animals of positive vehicle control group (3) display much lower 50% withdrawal thresholds of 2-3 grams. Comparison of Groups (4) to (6) shows a dose-dependent increase in the 50% withdrawal threshold to tactile stimuli. Consistent with the results of Example 16a, at a dose of 1 mg/kg, the Compound produces a moderate increase in threshold (a peak threshold of about 4 g at 4 hours, dropping to about 3 g at 6 hours). At a dose of 3 mg/kg, the increase in withdrawal threshold is significantly greater, rising to about 6 g at 1 hours, peaking at almost 8 g at 2 hours, then dropping to about 3 g at 4 hours. At a dose of 10 mg/kg, the increase in withdrawal threshold is much greater and reaches the range observed for the control animals. 10 mg/kg of Compound results in a threshold of about 9 g at 1 hour, peaking at about 10 g at 2 hours, then dropping to about 7 g at 4 hours and about 3 g at 6 hours.

### Example 16c: The Compound of Example 1 Suppresses Hyperalgesia in Response to Mechanical (Pressure) Stimuli

Rats are divided into six groups as described in Example 16a and are monitored for 6-hours after administration of the Compound of Example 1 or vehicle. This test is performed by measuring the static applied force threshold (pressure) for paw removal, as described above.

The results show that the control animals of both Group (1) and Group (2) display a withdrawal force threshold of 55 to 70 grams, which is within the normal range. In contrast, the animals of positive vehicle control group (3) display much lower withdrawal force threshold of 20-30 grams. Comparison of Groups (4) to (6) shows a dose-dependent increase in the withdrawal force threshold to mechanical stimuli. Consistent with the results of Example 16a and 16b, at a dose of 1 mg/kg, the Compound produces a moderate increase in threshold (a peak threshold of about 40 g at 4 hours, dropping to about 35 g at 6 hours). At a dose of 3 mg/kg, the increase in withdrawal threshold is greater, peaking at about 45 g at 2 hours, but then dropping to about 25 g at 6 hours. At a dose of 10 mg/kg, however, the increase in withdrawal threshold is much greater and more sustained, and reaches the range observed for the control animals. 10 mg/kg of Compound results in a threshold of about 60 g at 1 hour, dropping to 45-50 g at 2 hours to 4 hours, then dropping to about 35 g at 6 hours.

### Example 17: Animal Pharmacokinetic Data

Using standard procedures, the pharmacokinetic profile of the compound of Example 1 is studied in several animals.

### Example 17a: Rat PK Studies

In a first study, rats are administered the compound of Example 1 either by intravenous bolus (IV) at 1 mg/kg in 45% Trapposol vehicle, or orally (PO) at 10 mg/kg in 0.5% CMC vehicle (N=3 each group). In a second study, rats are administered the compound of Example 1 at 10 mg/kg PO or 3 mg/kg subcutaneously (SC), each in 45% Trapposol vehicle (N=6 for each group). Plasma concentrations of the drug are measured at time points from 0 to 48 hours post dose. Representative results are tabulated below (* indicates plasma concentration below measurable level of quantitation):

| | Study One | | Study Two | |
|---|---|---|---|---|
| | IV | PO | PO | SC |
| | (1 mg/kg) | (10 mg/kg) | (10 mg/kg) | (3 mg/kg) |
| 30 min (ng/mL) | 99.0 | 30.7 | 54.9 | 134.4 |
| 1 hour (ng/mL) | 47.3 | 37.2 | 60.6 | 140.9 |
| 6 hours (ng/mL) | 1.1 | 9.4 | 21.0 | 18.2 |
| 24 hours (ng/mL) | * | 0.1 | 0.4 | 1.9 |
| 48 hours (ng/mL) | * | * | ND | ND |
| Cmax (ng/mL) | 314.8 | 37.2 | 60.6 | 140.9 |
| AUC (ng-hr/mL) | 182 | 215 | 409 | 676 |
| Bioavailability | 100% | 12% | | |
| t-½ (hr) | | | 3.1 | 9.5 |

### Example 17b: Mice PK Studies

A similar study in mice is performed using 10 mg/kg PO administration of the compound of Example 1, and the following results are obtained: Tmax=0.25 hours; Cmax=279 ng/ml; AUC (0-4h)=759 ng-hr/mL; blood-plasma ratio (0.25-4 h) ranges from 3.7 to 6.6. The study is also conducted at a dose of 0.1 mg/kg SC. Representative results are shown in the table below:

| | Study: | | | |
|---|---|---|---|---|
| | PO, 10 mg/kg | | SC, 0.1 mg/kg | |
| | (0.5% CMC veh) | | (45% Trapposol veh) | |
| | Plasma | Brain | Plasma | Brain |
| Time (hr) | (ng/mL) | (ng/g) | (ng/mL) | (ng/g) |
| 0.25 | 279 | 1288 | 27.5 | 57.1 |
| 0.5 | 179 | 1180 | 31.1 | 71.9 |
| 1 | 258 | 989 | 29.2 | 78.5 |
| 2 | 153 | 699 | 14.6 | 38.7 |
| 4 | 199 | 734 | 4.7 | 32.6 |
| Tmax (hr) | 0.25 | 0.25 | 0.5 | 1.0 |
| Cmax | 279 | 1288 | 31.1 | 78.5 |
| (ng/mL) | | | | |
| AUC0-4 h | 759 | 2491 | 67 | 191 |
| (ng-hr/mL) | | | | |

| | | |
|---|---|---|
| B/P Ratio | 3.3 | 2.8 |

Together these results show that the compound of Example 1 is well-absorbed and distributed to the brain and tissues and is retained with a reasonably long half-life to enable once-daily administration of therapeutic doses.

### Example 18: Zucker Fatty Diabetic Rat Model of Neuropathic Pain

Insulin-resistant diabetes results spontaneously in the Zucker Fatty Diabetic (ZFD) rat. These rats display stable hyperglycemia and painful neuropathies which develop over several weeks. Painful neuropathies can be measured in rats using various tests, including assays for paw tactile and pressure responses and thermal (cold) thresholds. These tests can be used to measure the potential analgesic effects of therapies in development.

In these experiments, the effect of the Compound of Example 1 on pain thresholds in ZFD-diabetic rats is evaluated. The Compound of Example 1 is formulated for subcutaneous (s.c.) or intrathecal (i.t.) dosing in 10% Trappsol (beta-cyclodextrin) in water with addition of 1% Tween-80 to form a clear solution.

Forty (40) adult, male Sprague-Dawley rats weighing 225-275g at the start of the study are used in these experiments, including ten (10) lean, controls and thirty (30) ZFD rats. Animals are maintained in the vivarium at a controlled room temperature between 65 to 85° F. and a relative humidity between 30-70% under illumination by fluorescent lighting on a daily 12-hour light/dark cycle. All animals are maintained 2 per cage with free access to dry food and water.

Insulin-resistant diabetes is allowed to develop in male ZFD rats. Hypoglycemia is confirmed 4 days later in a sample of blood obtained by tail prick using a strip-operated reflectance meter, and is also confirmed at death. All animals are observed daily during the study period. Body weight and plasma glucose levels are determined at the end of the study.

Paw tactile response threshold: This test replicates the clinical assessment of allodynia to light touch as detected using von Frey filaments and serves as a standard assay for detection of allodynia developing within 2-4 weeks of diabetes appearance in ZFD-diabetic rats. The current method is described in detail by Calcutt, NC, Modeling Diabetic Sensory Neuropathy in Rats, METHODS IN MOLECULAR MEDICINE 99:55-65 (2004).

Paw pressure response threshold: This test applies greater force to the plantar hind paw than manual von Frey filaments and may be equated to pressure-induced pain such as that described by diabetic subjects upon standing and walking. Hyperalgesia to this measure develops over several weeks in ZFD rats such that this test can give an assessment of hyperalgesia and drug efficacy that differs from the allodynia measured by manual von Frey filaments. The method is described in detail by Lee-Kubil, Mixcoati-Zecuatl, Jolivalt, & Calcutt, Animal Models of Diabetes-Induced Neuropathic Pain, CURRENT TOPICS IN BEHAVIORAL NEUROSCIENCES 20:147-170 (2014).

Paw cold response threshold: This test replicates the clinical test of cold pain perception threshold. Rats are transferred to a testing cage with a wire mesh bottom and allowed to acclimate. A rigid filament attached to a Peltier thermoelectric cooling system is used to stimulate the plantar surface of the hind paw for 5 seconds. The stimulus is repeated 10 times at 2 to 5 minute intervals and the number of paw withdrawal responses is recorded and converted to a response frequency (%). This paradigm is repeated at various stimulation temperatures. ZFD diabetic rats develop cold hyperalgesia at temperatures of 10°° C. or less, while the normal response frequency above this temperature confirms that this does not represent an exaggerated response to applied pressure per se (see Table 18-1, below).

Paw formalin test: This test enables discrimination of pain driven by primary afferent input (phase 1) versus spinal sensitization and amplifications of primary afferent input (phase 2). There is, however, no clinical equivalent to this test. The method used here is described in detail in Calcutt (2004), supra.

Dosing procedures. Zucker Fatty Diabetic rats are found to develop hyperglycemia over 6-8 weeks. After confirmation of hyperglycemia, rats are tested weekly until baseline measurements showed onset of stable allodynia/hyperalgesia (3-6 weeks of diabetes). Upon confirmation of the presence of painful neuropathy, rats are tested for three measures of hyperalgesia: cold allodynia threshold, tactile hyperalgesia (von Frey filaments), and pressure responses measured by a mechanical von Frey device. All rats are tested in all three assays and all rats receive each of four (4) pre-treatments administered subcutaneously (s.c.) in a vehicle of 10% Trappsol in water. All rats receive treatments in the same order, as follows: vehicle, 1 mg/kg Compound of Ex. 1, 3 mg/kg Compound of Ex. 1, and 10 mg/kg Compound of Ex. 1. Rats receive pre-treatments 30 minutes prior to the start of testing. Hyperalgesia/allodynia measures are recorded from each rat at baseline (0 time), then 1, 2, 3, 4, and 6 h after treatment.

After the completion of testing of all rats at all dose/conditions for each assay (cold, tactile, and pressure responses) rats are out-fitted with cannulas for intrathecal (i.t.) application of vehicle or Compound of Ex. 1 (1, 3 or 10 μg) directly to spinal cord. All rats are then re-tested in each of the tree assays at each of the 4 pretreatment conditions/doses.

Statistical Analysis. Data for the cold allodynia test using s.c. dosing is analyzed using MANOVA for repeat measures over time conducted by group and baseline for each dose. A MANOVA is also conducted by dose and baseline for the ZFD rats+Compound of Ex. 1 groups (w/o vehicle for 0 dose level). A binary t-test is conducted for ZFD groups at each time point as well as binary t-tests of ZFD groups for change from baseline at each time point. Data for the manual von Frey test is analyzed using the Chi Square analysis for responders. For data from the electrical von Frey test, a one-way ANOVA is conducted across all groups, followed by MANOVA (repeat measures˜time) analysis by group and baseline for each dose and for the ZFD+Compound of Ex. 1 group (w/o vehicle for 0 dose level). A binary t-test was conducted for the ZFD group responses at each time point and for ZFD groups, change from baseline at each time point.

Cold Allodynia. ZFD rats_show a robust response frequency to paw presentation of a cold (10° C.) probe. Compound of Ex. 1 administered to rats in a 10% Trappsol vehicle (in water) given s.c. dose dependently reduces the response frequency (%) (Table 18-1) [“control” refers to the Sprague-Dawley lean control rats, and “ZFD” refers to the Zucker Fatty Diabetic rats]. The 3 and 10 mg/kg doses of Compound of Ex. 1 result in significant reductions in response frequency after treatment. Treatment of these rats with Compound of Ex. 1 i.t. also significantly reduces response frequency (%) at 1 and 3 μg doses; at 1 μg the difference from vehicle is significant whereas at 3 μg it is significant from vehicle at 1, 2, and 4h time points (Table 18-2).

TABLE 18-1
Cold Test (subcutaneous administration)

| | | Control + | | ZFD + | ZFD + | ZFD + |
|---|---|---|---|---|---|---|
| | | Cmpd. | | Cmpd. | Cmpd. | Cmpd. |
| Time | | Ex 1 | | Ex 1 | Ex 1 | Ex 1 |
| (hr) | Control | (10 mg/kg) | ZFD | (1 mg/kg) | (3 mg/kg) | (10 mg/kg) |
| 0 | 10 | 16 | 91 | 88 | 88 | 90 |
| 1 | 11 | 18 | 93 | 96 | 32 | 28 |
| 2 | 11 | 10 | 95 | 94 | 26 | 16 |
| 4 | 14 | 10 | 90 | 86 | 90 | 48 |

TABLE 18-2
Cold Test (intrathecal administration)

| | | Control + | | ZFD + | ZFD + | |
|---|---|---|---|---|---|---|
| | | Cmpd. | | Cmpd. | Cmpd. | ZFD + |
| Time | | Ex 1 | | Ex 1 | Ex 1 | Cmpd. |
| (hr) | Control | (10 μg) | ZFD | (1 μg) | (3 μg) | Ex 1 (10 μg) |
| 0 | 9 | 17 | 87 | 93 | 77 | 93 |
| 1 | 10 | 11 | 74 | 90 | 37 | 33 |
| 2 | 14 | 20 | 86 | 83 | 27 | 33 |
| 4 | 12 | 14 | 92 | 83 | 70 | 77 |

Manual von Frey (tactile) ZFD rats show a robust response of paw presentation of manual von Frey filaments as measured by a decrease in the threshold for paw withdrawal. Compound of Ex. 1 administered to rats in a 10% Trappsol vehicle (in water), given s.c., dose dependently reduces the threshold for paw withdrawal (Table 18-3). The 3 mg/kg dose of Compound of Ex. 1 results in significant normalization of the threshold for paw withdrawal that is evident at 1 and 2 h after treatment; the 10 mg/kg dose elicits a significant difference from vehicle at 1, 2, and 4 h time points. Treatment of these rats with Compound of Ex. 1 i.t. also significantly normalizes thresholds at the 1 μg dose level (Table 18-4).

TABLE 18-3
Manual von Frey (subcutaneous administration)

| | | Control + | | ZFD + | ZFD + | ZFD + |
|---|---|---|---|---|---|---|
| | | Cmpd. | | Cmpd. | Cmpd. | Cmpd. |
| Time | | Ex 1 | | Ex 1 | Ex 1 | Ex 1 |
| (hr) | Control | (10 mg/kg) | ZFD | (1 mg/kg) | (3 mg/kg) | (10 mg/kg) |
| 0 | 14.35 | 13.98 | 1.99 | 3.11 | 2.22 | 1.71 |
| 1 | 13.77 | 12.28 | 2.38 | 2.61 | 6.48 | 6.97 |
| 2 | 14.57 | 13.93 | 1.88 | 1.70 | 10.15 | 13.47 |
| 4 | 14.67 | 13.45 | 1.83 | 2.07 | 2.88 | 9.25 |

TABLE 18-4
Manual von Frey (intrathecal administration)

| | | Control + | | ZFD + | ZFD + | ZFD + |
|---|---|---|---|---|---|---|
| | | Cmpd. | | Cmpd. | Cmpd. | Cmpd. |
| Time | | Ex 1 | | Ex 1 | Ex 1 | Ex 1 |
| (hr) | Control | (10 μg) | ZFD | (1 μg) | (3 μg) | (10 μg) |
| 0 | 13.26 | 11.90 | 1.48 | 2.06 | 5.78 | 1.92 |
| 1 | 13.08 | 13.90 | 2.73 | 2.28 | 9.68 | 8.53 |
| 2 | 13.21 | 14.14 | 1.77 | 3.32 | 12.11 | 11.57 |
| 4 | 13.49 | 13.67 | 1.77 | 4.25 | 7.01 | 4.77 |

Paw Pressure Response. ZFD rats show a robust response frequency to paw presentation of mechanical von Frey filaments. Compound of Ex. 1 administered to rats in a 10% Trappsol vehicle (in water) given s.c. dose dependently reduces the response frequency (%) (Table 18-5). The 3 mg/kg dose of Compound of Ex. 1 results in significant reductions in response frequency at 1 and 2 h after treatment; 10 mg/kg elicits significant improvement in pain responses at 1, 2, 4, and 6 h time points. Treatment of these rats with Compound of Ex. 1 i.t. also significantly reduces response frequency (%) at 1 μg (2 and 4 h) and at 3 μg (1, 2, and 4 h) compared with vehicle (Table 18-6).

TABLE 18-5
Electronic von Frey (subcutaneous administration)

| | | Control + | | ZFD + | ZFD + | ZFD + |
|---|---|---|---|---|---|---|
| | | Cmpd. | | Cmpd. | Cmpd. | Cmpd. |
| Time | | Ex 1 | | Ex 1 | Ex 1 | Ex 1 |
| (hr) | Control | (10 mg/kg) | ZFD | (1 mg/kg) | (3 mg/kg) | (10 mg/kg) |
| 0 | 62.44 | 58.64 | 23.88 | 28.36 | 33.72 | 22.69 |
| 1 | 63.83 | 55.73 | 23.27 | 32.99 | 54.21 | 56.02 |
| 2 | 58.72 | 60.74 | 22.08 | 27.35 | 58.41 | 58.15 |
| 4 | 64.37 | 62.89 | 23.11 | 24.67 | 30.01 | 39.32 |

TABLE 18-6
Electronic von Frey (intrathecal administration)

| | | Control + | | ZFD + | ZFD + | |
|---|---|---|---|---|---|---|
| | | Cmpd. | | Cmpd. | Cmpd. | ZFD + |
| Time | | Ex 1 | | Ex 1 | Ex 1 | Cmpd. |
| (hr) | Control | (10 μg) | ZFD | (1 μg) | (3 μg) | Ex 1 (10 μg) |
| 0 | 62.49 | 63.68 | 26.88 | 24.76 | 33.23 | 24.68 |
| 1 | 62.52 | 60.91 | 24.32 | 34.77 | 53.94 | 46.37 |
| 2 | 59.92 | 62.10 | 24.43 | 30.09 | 50.17 | 45.01 |
| 4 | 61.51 | 59.56 | 24.75 | 28.19 | 39.95 | 32.33 |

Formalin Test. Compound of Ex. 1, given either s.c. or i.t., has no effect on pain responses in either early or late phase of the formalin test, when administered at the end of the study.

Zucker Fatty Diabetic rats develop stable and robust painful neuropathic pain responses that persist for months. These rats exhibit significant increases in cold allodynia as well as reduced pain thresholds in tests of tactile and pressure hyperalgesia assays. The Compound of Ex. 1, free base, given either s.c. or i.t., significantly attenuated painful neuropathy in all three tests. The showing that the similar results are obtained s.c. and i.t. demonstrates that the effect is not merely a peripherally mediated effect. The data support the conclusion that Compound of Ex. 1 attenuates painful neuropathic pain response in rats sustaining insulin-deficient diabetes.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A method for the treatment of neuropathic pain, comprising administering to a patient in need thereof a compound of Formula I: 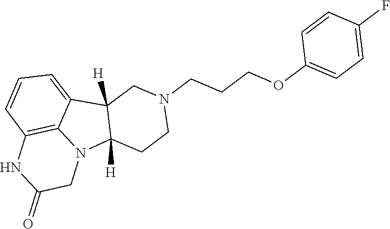 [SMILES: O=C1CN2c3c(cccc3[C@@H]3CN(CCCOc4ccc(F)cc4)CC[C@@H]32)N1]
in free or pharmaceutically acceptable salt form;
wherein the neuropathic pain is caused by a peripheral neuropathy or is caused by a central neuropathy.

18. The method according to claim 17, comprising the compound of Formula I in the form of a pharmaceutically acceptable salt.

19. The method according to claim 17, wherein the compound of Formula I is administered in the form of a pharmaceutical composition comprising the compound of Formula I in admixture with a pharmaceutically acceptable diluent or carrier.

20. The method according to claim 19, wherein the pharmaceutical composition is a sustained release or delayed release formulation.

21. The method according to claim 19, wherein the pharmaceutical composition comprises the Compound of Formula I in a polymeric matrix.

22. (canceled)

23. The method according to claim 17, wherein the peripheral neuropathy is a multiple mononeuropathy or a polyneuropathy.

24. The method according to claim 17, wherein the patient was previously treated with another pain-relieving medication, and the patient did not respond adequately to said medication.

25. (canceled)

26. (canceled)

27. The method according to claim 23, wherein the polyneuropathy is a diabetic polyneuropathy.

28. The method according to claim 17, wherein the peripheral neuropathy is a mononeuropathy.

29. The method according to claim 17, wherein the peripheral neuropathy is a focal mononeuropathy, a pressure mononeuropathy, or an entrapment mononeuropathy.

30. The method according to claim 17, wherein the peripheral neuropathy is carpal tunnel syndrome.

31. The method according to claim 17, wherein the peripheral neuropathy is a radiculopathy.

32. The method according to claim 31, wherein the radiculopathy is caused by diabetic ischemia.

33. The method according to claim 17, wherein the peripheral neuropathy is a plexopathy.

34. The method according to claim 33, wherein the plexopathy is caused by nerve compression.

35. The method according to claim 17, wherein the central neuropathy is a deafferentation pain or a sympathetically maintained pain.

36. The method according to claim 35, wherein the central neuropathy is complex regional pain syndrome (CRPS).

37. The method according to claim 17, wherein the peripheral neuropathy or central neuropathy is caused by a drug-induced neurotoxicity.

38. The method according to claim 37, wherein the drug-induced neurotoxicity is caused by doxorubicin, etoposide, gemcitabine, ifosfamide, interferon alfa, cisplatin, carboplatin, oxaliplatin, nedaplatin, triplatin, phenanthriplatin, picoplatin, satraplatin, vinblastine, vincristine, vindesine, vinorelbine, vinpocetin, didanosine, stavudine, or zalcitabine.

39. The method according to claim 17, wherein the peripheral neuropathy or central neuropathy is caused by postherpetic neuralgia (PHN) or by fibromyalgia.

40. The method according to claim 17, wherein the compound of Formula I is in acid addition salt form, wherein the acid is hydrochloric, toluenesulfonic, glutamic, tartaric, malic or ascorbic acid.
